(19) United States
(12) Patent Application Publication
(10) Pub. No.: US 2025/0186396 A1
(43) Pub. Date: Jun. 12, 2025

(54) COMPOSITIONS AND METHODS FOR THE TREATMENT OF GLIOBLASTOMA WITH A DUAL TARGETING ANTI-TUMOR COMPOSITION

(71) Applicant: Northwestern University, Evanston, IL (US)

(72) Inventors: Yang LIU, Evanston, IL (US); Peiwen CHEN, Evanston, IL (US)

(21) Appl. No.: 18/970,577

(22) Filed: Dec. 5, 2024

Related U.S. Application Data
(60) Provisional application No. 63/606,528, filed on Dec. 5, 2023.

(51) Int. Cl. A61K 31/4025; A61K 31/275; A61K 31/381; A61K 45/06; A61P 35/00

(52) U.S. Cl. CPC A61K 31/4025; A61K 31/275; A61K 31/381; A61K 45/06; A61P 35/00

(57) ABSTRACT

The present disclosure provides compositions and methods for the treatment of cancer (e.g., glioblastoma). In particular, the present disclosure provides a dual targeting anti-tumor composition comprising a LOX inhibitor and a CLOCK-OLFML3 inhibitor, and methods of use thereof for treating glioblastoma in combination with or without an anti-programmed death-1 (PD1) therapy or radiotherapy.

### CROSS-REFERENCE TO RELATED APPLICATIONS

This application claims the benefit of U.S. Provisional Patent Application No. 63/606,528, filed on Dec. 5, 2024, which is incorporated by reference herein.

### STATEMENT REGARDING FEDERALLY SPONSORED RESEARCH OR DEVELOPMENT

This invention was made with government support under grant numbers CA240896, NS124594 and NS127824 awarded by the National Institutes of Health and grant number W81XWH-21-1-0380 awarded by the U.S. Army Medical Research and Development Command. The government has certain rights in the invention.

### SEQUENCE LISTING

The text of the computer readable sequence listing filed herewith, titled “NWEST_42559_202_SequenceListing.xml”, created Mar. 5, 2025, having a file size of 32,756 bytes, is hereby incorporated by reference in its entirety.

### FIELD

The present disclosure provides compositions and methods for the treatment of cancer (e.g., glioblastoma). In particular, the present disclosure provides a dual targeting anti-tumor composition comprising a LOX inhibitor and a CLOCK-OLFML3 inhibitor, and methods of use thereof for treating glioblastoma in combination with or without an anti-programmed death-1 (PD1) therapy or radiotherapy.

### BACKGROUND

Glioblastoma (GBM) is an aggressive, fast-growing brain tumor that typically originates in the cerebral hemispheres. It is characterized by its rapid cell division and invasive growth pattern, allowing it to infiltrate surrounding brain tissue and stimulate the formation of new blood vessels through angiogenesis. Common symptoms of GBM include headaches, seizures, altered mood, shifts in cognitive function, physical weakness, and visual disturbances.

Despite advancements in understanding GBM's underlying mechanisms, patient prognosis remains poor, with a median survival of only 15-20 months post-diagnosis. Current GBM treatment involves maximal surgical resection, followed by radiation therapy and concurrent chemotherapy. However, these treatments, when prolonged, can lead to treatment resistance and adverse side effects.

In-depth genomic studies have unveiled alterations in oncogenes and tumor suppressor genes as driving factors in certain GBM cases. These findings hold significance as they point to emerging evidence suggesting that abnormal molecular activities associated with cancer extend beyond cancer cells to involve stromal cells within the tumor microenvironment (TME).

A distinguishing characteristic of the GBM TME is the high abundance of tumor-associated macrophages and microglia (TAMs), constituting up to 50% of the total cells in the tumor mass. TAMs play a dual role in the TME, with potential benefits and detriments. They can influence the immune response and facilitate tumor progression, as well as modulate the immunosuppressive microenvironment.

Other cancer treatments have explored the use of immunotherapies like immune checkpoint inhibitors (ICIs) with modest improvement patients. However, emerging evidence demonstrates that such ICI therapies only produce modest clinical benefit in GBM patients due to the presence of highly immunosuppressive cells (e.g., TAMs) in the TME. Likewise, genomic and transcriptomic analyses have revealed that TAMs, along with PTEN mutations, can contribute to anti-PD1 therapy resistance in GBM. Moreover, TAMs have a detrimental effect on the response to traditional cancer treatments like radiotherapy. These findings support the importance of TAMs in affecting therapy resistance; however, there is no effective therapeutic approach to target them in the GBM TME. Understanding the complex interactions of TAMs in the TME is crucial for developing more effective treatment strategies for glioblastoma.

### SUMMARY

The present disclosure provides compositions and methods for the treatment of cancer (e.g., glioblastoma). In particular, the present disclosure provides a dual targeting anti-tumor composition comprising a LOX inhibitor and a CLOCK-OLFML3 inhibitor, and methods of use thereof for treating glioblastoma in combination with or without an anti-programmed death-1 (PD1) therapy or radiotherapy.

Embodiments of the present disclosure include a dual targeting anti-tumor composition comprising a LOX inhibitor and a CLOCK-OLFML3 inhibitor. In some embodiments, the LOX inhibitor suppresses macrophage infiltration and upregulates microglial infiltration. In some embodiments, the LOX inhibitor upregulates expression of Programmed Death-Ligand 1 (PD-L1) and OLFML3. In some embodiments, the LOX inhibitor modulates OLFML3 expression via the NF-kB-PATZ1 signaling axis. In some embodiments, the LOX inhibitor is a β-aminopropionitrile (BAPN), BAPN derivative, or an aminomethylenethiophene (AMT). In some embodiments, the LOX inhibitor is an inhibitor of LOX expression.

In some embodiments, the CLOCK-OLFML3 inhibitor suppresses microglial infiltration. In some embodiments, the CLOCK-OLFML3 inhibitor suppresses OLFML3 expression. In some embodiments, the CLOCK-OLFML3 inhibitor is a SR9009. In some embodiments, the CLOCK-OLFML3 inhibitor is an inhibitor of CLOCK-OLFML3 expression.

In other embodiments, the LOX inhibitor and the CLOCK-OLFML3 inhibitor are administered simultaneously or independently.

Further provided herein are methods for treating cancer in a subject comprising administering a dual targeting anti-tumor composition.

In some embodiments, provided herein are methods for treating cancer in a subject comprising administering an effective amount of the dual targeting anti-tumor composition with an effective amount of a cancer therapeutic to a subject in need of treatment thereof.

In some embodiments, further comprising administering the dual targeting anti-tumor composition and the cancer therapeutic simultaneously or independently.

In some embodiments, the cancer is glioblastoma (GBM).

In some embodiments, the GBM is PTEN-deficient.

In some embodiments, the cancer therapeutic is an Anti-Programmed Death 1 (PD1) therapy. In other embodiments, the cancer therapeutic is a radiotherapy.

In some embodiments, the subject is a human.

In some embodiments, the manufacture of a medicament for the treatment of cancer in a subject in need thereof.

### BRIEF DESCRIPTION OF THE DRAWINGS

FIGS. 1A-K. LOX inhibition improves the efficacy of anti-PD1 therapy. (A) High-resolution uniform manifold approximation and projection (UMAP) dimensional reduction of different subtypes, including mesenchymal-like (MES-like), neural-progenitor-like (NPC-like), astrocyte-like (AC-like) and oligodendrocyte-progenitor-like (OPC-like), of tumor cells from GBM patient tumors. (B) Pattern representing single-cell gene expression of LOX in distinct subtypes of tumor cells. (C) Percentage MES-like GBM cells out of total GBM cells, and normalized LOX gene expression in different subtypes of malignant cells in GBM patient tumors. (D) Activated CD8⁺ T cell signature in different subtypes of GBM tumors. (E) Gene Set Enrichment Analysis (GSEA) analysis for various types of immune cells in LOX-high and LOX-low patient tumors from the Cancer Genome Atlas (TCGA) GBM database. Immunofluorescence (F) and quantification (G) of relative CD8⁺CD69⁺ T cells in tumors from CT2A tumor-bearing mice treated with or without LOX inhibitor BAPN (2 g/L in drinking water) on day 4. Scale bar, 50 μm. n=3 independent samples. Student's t test. Immunofluorescence (H) and quantification (I) of relative CD4⁺ cells in tumors from CT2A-bearing mice treated with or without BAPN (2 g/L in drinking water) on day 4. Scale bar, 50 μm. n=3 independent samples. Student's t test. (J and K) Survival curves of C57BL/6 mice implanted with CT2A cells (2×10⁴ cells/mouse, J) or 005 GSCs (2×10⁵ cells/mouse, K). Mice were treated with BAPN (2 g/L in drinking water) on day 4, and then received the treatment with IgG or anti-PD1 (10 mg/kg body weight, i.p.) on days 11, 14, and 17. n=5-7 mice per group. Log-rank test. *, P<0.05, **, P<0.01, ***, P<0.001.

FIGS. 2A-I. Macrophages suppress microglia infiltration into the GBM TME. (A) High-resolution UMAP dimensional reduction of myeloid cells, including macrophages, microglia, monocytes (Mono), dendritic cells (DCs), and myeloid-derived suppressor cells (MDSCs), from GBM patient tumors. (B) Percentage of different types of myeloid cells in tumors of low-grade gliomas (LGG), newly diagnosed GBM (ndGBM) and recurrent GBM (rGBM). The molecular features of this tumor were ATRX (alpha-thalassemia/mental retardation, X-linked) retained with mutations in PTEN, TERT (Telomerase Reverse Transcriptase), and RB1 (Retinoblastoma 1) with amplification of EGFR (Epidermal Growth Factor Receptor) and MDM4 (Mouse Double Minute 4). (C) Correlation between macrophages and microglia in the GBM TME. Pearson test. (D) Representative image of multiplex sequential immunofluorescence showing the distribution of P2RY12⁺ microglia, CD163⁺ macrophages, GFAP⁺ tumor cells and CD31⁺ blood vessels in the tumor edges and tumors from IDH1-WT GBM patients. Scale bar, 500 μm. (E and F) Higher magnified view of CD163⁺ macrophages and P2RY12⁺ microglia in the tumor edges and tumors from IDH1-WT GBM patients. Scale bar, 100 μm. Schematic diagram (G), representative images (H) and quantification (I) of relative migration of HMC3 microglia following stimulation with THP-1 macrophages or U87 conditioned media (CM)-educated THP-1 macrophages (T-macrophage). Scale bar, 400 μm. n=3 independent samples. Student's t test. **, P<0.01, ***, P<0.001.

FIGS. 3A-V. LOX inhibition increases OLFML3 expression and microglia infiltration in GBM. (A) Identification of four overlapping PTEN-LOX axis-regulated genes encoding secreted factors in PTEN-knockout (KO) versus WT SF763 cells and in LOX shRNA (shLOX) versus shRNA control (shC) U87 cells. (B) Heatmap representation of the expression of these four identified genes in PTEN-KO and WT SF763 cells as well as shC and shLOX U87 cells. Red signal indicates higher expression, and blue signal denotes lower expression. Immunoblots for OLFML3 in lysates of U87 cells (C) and PTEN-KO SF763 cells (D) expressing shC and shLOX. (E) Immunoblots for OLFML3 in lysates of U87 cells and PTEN-KO SF763 cells treated with BAPN at indicated concentrations. (F) Immunoblots for OLFML3 in lysates of CT2A cells and 005GSC cells treated with BAPN at indicated concentration. Immunofluorescence (G) and quantification (H) of relative CX3CR1⁺ microglia in tumors from CT2A-bearing mice treated with or without BAPN (2 g/L in drinking water) on day 4. Scale bar, 50 μm. n=3 independent samples. Student's t test. Representative images (I) and quantification (J) of flow cytometry for the percentage of intratumoral CD45^{low}WCD11b⁺CX3CR1⁺ microglia in size matched tumors from CT2A tumor-bearing mice treated with or without BAPN (2 g/L in drinking water) on day 4. n=3 independent samples. Student's t test. Survival curves of C57BL/6 mice implanted with CT2A cells (2×10⁴ cells/mouse) (K) or 005 GSCs (2×10⁵ cells/mouse) (L). Mice were treated with BAPN (2 g/L in drinking water) on day 4, and then received the treatment with SR9009 (100 mg/kg body weight, i.p.) daily for 10 days beginning at day 7 post-orthotopic injection. n=5-7 mice for each group. Log-rank test. (M) Representative of immunofluorescence staining of Ki67 and cleaved caspase 3 (CC3) in tumors from CT2A-bearing mice treated with or without BAPN and SR9009. Scale bars, 50 μm. Quantification of the relative expression levels of Ki67 (N) and CC3 (O) in tumors from CT2A-bearing mice treated with or without BAPN and SR9009. n=3 independent samples. One-way ANOVA test. Representative images (P) and quantification (Q) of relative migration of HMC3 microglia following stimulation with the conditioned media (CM) from U87 cells pretreated with or without BAPN (200 μM). Scale bar, 400 μm. n=3 independent samples. Student's t test. (R) Immunoblots for OLFML3 and LOX in lysates of GL261 cells in the presence or absence of LOX overexpression (OE). (S-V) Immunofluorescence(S) and quantification of relative F4/80⁺ macrophages (T), CX3CR1⁺ microglia (U), and OLFML3⁺ cells (V) in tumors from mice implanted with control and LOX-overexpressed GL261 cells. Scale bar, 50 μm. n=3 independent samples. Student's t test. *, P<0.05, **, P<0.01, ***, P<0.001.

FIGS. 4A-L. LOX regulates OLFML3 expression through regulating the NF-κB-PATZ1 signaling axis. (A) GSEA analysis on RNA-seq data of U87 cells with LOX shRNA knockdown (shLOX) versus shRNA control (shC) shows top 10 enriched oncogenic signaling pathways. (B) Immunoblots for P-P65, P65, and LOX in lysates of U87 and PTEN-KO SF763 cells expressing shC and shLOX. (C) Relative mRNA expression of OLFML3 in PTEN-KO SF763 cells expressing shC and shLOX treated with or without P65 inhibitor (P65i) SC75741 (5 μM). n=3 independent samples. Student's t test. (D) Identification of 22 overlapping transcription factors (TFs) in TCGA GBM tumors (LOX-low versus-high) and U87 cells (shLOX versus shC). (E) Relative mRNA expression of 10 TFs in PTEN-KO SF763 cells expressing shC and shLOX. n=3 independent samples. One-way ANOVA test. (F) Relative mRNA expression of the 10 TFs in U87 cells treated with or without LOX inhibitor BAPN (200 μM). n=3 independent samples. Student's t test. (G) Immunoblots for PATZ1 (POZ/BTB and AT-hook-containing Zinc Finger Protein 1) in lysates of PTEN-KO SF763 cells expressing shC and shLOX. (H) Relative mRNA expression of PATZ1 in PTEN-KO SF763 cells expressing shC and shLOX and treated with or without P65i SC75741 (5 μM). n=3 independent samples. Student's t test. (I) Schematic of designing Chromatin Immunoprecipitation (ChIP)-qPCR primers based on 3 potential binding sites. (J) Quantification of PATZ1 ChIP-PCR in the OLFML3 promoter of PTEN-KO SF763 cells. IgG was used as the Control. n=3 independent samples. Student's t test. (K) Immunoblots for OLFML3 in lysates of PTEN-KO SF763 cells with or without PATZ1 overexpression (OE) and treated with or without P65 activator (+). (L) Immunoblots for OLFML3 in lysates of PTEN-WT SF763 cells expressing shC and shPATZ1 treated with or without P65i SC75741 (5 μM). n.s., not significant, P>0.05, *, P<0.05, **, P<0.01, ***, P<0.001.

FIGS. 5A-R. Dual Inhibition of LOX and CLOCK-OLFML3 axis activates anti-tumor immune response and synergizes with anti-PD1 therapy. (A and B) Immunofluorescence (A) and quantification (B) of relative CD8⁺CD69⁺ T cells in tumors from CT2A model (2×10⁴ cells/mouse) treated with or without BAPN (2 g/L in drinking water) on day 4, and/or SR9009 (100 mg/kg/day, i.p.) for 10 days beginning at day 7 post-orthotopic injection. Scale bar, 50 μm. n=3 independent samples. One-way ANOVA test. (C and D) Immunofluorescence (C) and quantification (D) of relative CD4⁺ T cells in tumors from CT2A model (2×10⁴ cells/mouse) treated with or without BAPN (2 g/L in drinking water) on day 4, and SR9009 (100 mg/kg body weight, i.p.) daily for 10 days beginning at day 7 post-orthotopic injection. Scale bar, 50 μm. n=3 independent samples. One-way ANOVA test. (E and F) Survival curves of C57BL/6 mice implanted with CT2A cells (2×10⁴ cells/mouse, E) or 005 GSCs (2×10⁵ cells/mouse, F). Mice were treated with BAPN (2 g/L in drinking water) on day 4, SR9009 (100 mg/kg body weight, i.p.) daily for 10 days beginning at day 7 post-orthotopic injection, and anti-PD1 (10 mg/kg body weight, i.p.) on days 11, 14, and 17. n=7-10 mice for each group. Log-rank test. (E and F). (G and H) Representative images (G) and quantification (H) of flow cytometry for the percentage of intratumoral CD8⁺CD69⁺ T cells in size matched tumors from CT2A tumor-bearing mice treated with or without BAPN (2 g/L in drinking water) on day 4, and/or SR9009 (100 mg/kg/day, i.p.) for 10 days beginning at day 7 post-orthotopic injection. n=3 independent samples. One-way ANOVA test. (I and J) Representative images (I) and quantification (J) of flow cytometry for the percentage of intratumoral CD8⁺IFN-γ⁺ T cells in size matched tumors from CT2A tumor-bearing mice treated with or without BAPN (2 g/L in drinking water) on day 4, and/or SR9009 (100 mg/kg/day, i.p.) for 10 days beginning at day 7 post-orthotopic injection. n=3 independent samples. One-way ANOVA test. (K and L) Representative images (K) and quantification (L) of flow cytometry for the percentage of intratumoral CD8⁺CD69⁺ T cells in size matched tumors from 005GSC tumor-bearing mice treated with or without BAPN (2 g/L in drinking water) on day 4, and/or SR9009 (100 mg/kg/day, i.p.) for 10 days beginning at day 7 post-orthotopic injection. n=3 independent samples. One-way ANOVA test. (M and N) Representative images (M) and quantification (N) of flow cytometry for the percentage of intratumoral CD8⁺IFN-γ⁺ T cells in size matched tumors from 005GSC tumor-bearing mice treated with or without BAPN (2 g/L in drinking water) on day 4, and/or SR9009 (100 mg/kg/day, i.p.) for 10 days beginning at day 7 post-orthotopic injection. n=3 independent samples. One-way ANOVA test. (O and P) Survival curves of C57BL/6 mice implanted with CT2A cells (2×10⁴ cells/mouse, O) or 005 GSCs (2×10⁵ cells/mouse, P). Mice were treated with SR9009 (100 mg/kg/day, i.p.) for 10 days beginning at day 7 post-orthotopic injection and then received the treatment with IgG or anti-PD1 (10 mg/kg, i.p.) on days 11, 14, and 17. n=5 mice per group. Log-rank test. (Q and R) Cured mice from the triple therapy were re-challenged on day 70 with CT2A cells (2×10⁴ cells/mouse, Q) or on day 110 with 005 GSCs (2×10⁵ cells/mouse, R). Similarly aged naive mice were implanted as controls. n=5 mice per group. Log-rank test. *, P<0.05, **, P<0.01, ***, P<0.001.

FIGS. 6A-M. Dual targeting macrophages and microglia enhances anti-tumor efficiency of radiation therapy. Immunofluorescence (A) and quantification (B) of relative F4/80⁺ macrophages in tumors from CT2A model (2×10⁴ cells/mouse) treated with or without brain radiation (2.5 Gy) for 4 days beginning at day 7 post-orthotopic injection. Scale bar, 50 μm. n=3 independent samples. Student's t test. Immunofluorescence (C) and quantification (D) of relative CX3CR1⁺ microglia in tumors from CT2A model (2×10⁴ cells/mouse) treated with or without brain radiation (2.5 Gy) for 4 days beginning at day 7 post-orthotopic injection. Scale bar, 50 μm. n=3 independent samples. Student's t test. Immunoblots for CLOCK, OLFML3, and LOX in lysates of U87 cells (E), PTEN-KO SF763 cells (F), and CT2A cells (G) received radiation at indicated doses. Survival curves of C57BL/6 mice implanted with CT2A cells (2×10⁴ cells/mouse, H) or 005 GSCs (2×10⁵ cells/mouse, I). Mice were treated with BAPN (2 g/L in drinking water) on day 4, and SR9009 (100 mg/kg body weight, i.p.) daily for 10 days beginning at day 7 post-orthotopic injection, concurrent with or without radiation treatment (2.5 Gy) for 4 days beginning at day 7 post-orthotopic injection. n=5-11 mice for each group. Log-rank test. Immunofluorescence (J) and quantification (K) of Ki67 level in tumors from the brains of mice intracranially implanted with CT2A cells (2×10⁴ cells/mouse) and received the indicated treatments. Scale bar, 50 μm. n=3 independent samples. One-way ANOVA test. Immunofluorescence (L) and quantification (M) of cleaved caspase 3 (CC3) level in tumors from the brains of mice intracranially implanted with CT2A cells (2×10⁴ cells/mouse) and received the indicated treatments. Scale bar, 50 μm. n=3 independent samples. One-way ANOVA test. *, P<0.05, **, P<0.01, ***, P<0.001.

FIGS. 7A-R. Inhibition of LOX increases intratumoral T cell infiltration and upregulates PD-L1 expression in PTEN-deficient GBM cells. Immunofluorescence (A) and quantification (B) of relative CD8⁺ cells in tumors from 005 GSC-bearing mice treated with IgG or LOX neutralizing antibodies (Ab, 20 mg/kg, i.p., once every 4 days) starting at 4 days post-orthotopic injection. Scale bar, 100 μm. n=3 independent samples. Student's t test. Immunofluorescence (C) and quantification (D) of relative CD4⁺ cells in tumors from 005 GSC-bearing mice treated with IgG or LOX Ab (20 mg/kg, i.p., once every 4 days) starting at 4 days post-orthotopic injection. Scale bar, 100 μm. n=3 independent samples. Student's t test. Immunoblots for PD-L1 in lysates of U87 cells (E) and PTEN-KO SF763 cells (F) expressing shRNA control (shC) and LOX shRNAs (shLOX). (G) Immunoblots for PD-L1 in lysates of U87 cells and PTEN-KO SF763 cells treated with BAPN at indicated concentrations. (H) Immunoblots for PD-L1 in lysates of CT2A cells and 005 GSCs treated with BAPN at indicated concentration. (I-K) The concentration of BAPN in the plasma (I and K) and brain (J and K) of mice after drug administration at indicated time points. (L and M) Immunofluorescence images of staining controls using secondary Alexa Fluor® 594 Conjugate (L) and Alexa Fluor® 488 Conjugate antibodies (M). Scale bar, 50 μm. (N and O) Immunoblots for PD-L1 and LOX in lysates of GSC23 (N) and GSC7-10 (O) expressing shRNA control (shC) and LOX shRNAs (shLOX). (P) Immunoblots for PD-L1 in lysates of GSC23 and GSC7-10 treated with BAPN at indicated concentrations. (Q and R) Immunofluorescence (Q) and quantification (R) of relative CD8⁺ T cells in tumors from CT2A model (2×10⁴ cells/mouse) treated with or without LOX inhibitor BAPN (2 g/L in drinking water) on day 4. Scale bar, 50 μm. n=3 independent samples. Student's t test.

FIGS. 8A-H. Relationship between macrophages and microglia in the GBM TME. Percentage of different myeloid cell cells in tumors of newly diagnosed GBM (ndGBM, A) and recurrent GBM (rGBM, B) from the single cell RNA-sequencing dataset. Higher magnified view of CD31⁺ blood vessels, P2RY12⁺ microglia and CD163⁺ macrophages in tumors (C) and tumor edges (D) from IDH1-WT GBM patients. Scale bar, 50 μm. Schematic diagram (E), representative images (F) and quantification (G) of relative migration of THP-1 macrophages following stimulation with HMC3 microglia or U87 conditioned media (CM)-educated microglia (T-microglia). Scale bar, 200 μm. n=3 independent samples. One-way ANOVA test. (H) Representative images of H&E staining to designate the tumor edges from IDH1-WT GBM patients. Scale bar, 5 mm (left), 250 μm (right upper), and 500 μm (right bottom). n.s., not significant, P>0.05.

FIGS. 9A-Z. LOX inhibition reduces macrophage infiltration but promotes microglia infiltration in GBM. Representative images (A) and quantification (B) of relative migration of HMC3 microglia following stimulation with conditioned media (CM) from U87 cells expressing control shRNA (shC) and LOX shRNAs (shLOX). Scale bar, 400 μm. n=3 independent samples. One-way ANOVA test. Immunofluorescence (C) and quantification (D) of relative F4/80⁺ macrophages in tumors from CT2A-bearing mice treated with or without BAPN (2 g/L in drinking water) on day 4. Scale bar, 50 μm. n=3 independent samples. Student's t test. Representative images (E) and quantification (F) of flow cytometry for the percentage of intratumoral CD45^{high}CD11b⁺CD68⁺ macrophages in size matched tumors from CT2A-bearing mice treated with or without BAPN (2 g/L in drinking water) on day 4. n=3 independent samples. Student's t test. Immunofluorescence (G) and quantification (H) of relative CX3CR1⁺ microglia in tumors from CT2A-bearing mice treated with or without SR9009 (100 mg/kg body weight, i.p.) daily for 10 days beginning at day 7 post-orthotopic injection. Scale bar, 50 μm. n=3 independent samples. Student's t test. Immunofluorescence (I) and quantification (J) of relative F4/80⁺ macrophages in tumors from CT2A-bearing mice treated with or without SR9009 (100 mg/kg body weight, i.p.) daily for 10 days beginning at day 7 post-orthotopic injection. Scale bar, 50 μm. n=3 independent samples. Student's t test. Immunoblots for LOX and CLOCK in lysates of QPP7 GSCs (K) and CT2A cells (L) expressing shC and shClock. (M) Immunoblots for LOX in lysates of CT2A cells and 005 GSCs treated with SR9009 (5 μM). (N and O) Immunoblots for OLFML3 and LOX in lysates of GSC23 (N) and GSC7-10 (O) expressing shRNA control (shC) and LOX shRNAs (shLOX). (P) Immunoblots for OLFML3 in lysates of GSC23 and GSC7-10 treated with BAPN at indicated concentrations. (Q and R) Representative images (Q) and quantification (R) of relative migration of SIM-A9 microglia following stimulation with the CM from control and LOX-overexpressed GL261 cells. Scale bar, 200 μm. n=3 independent samples. One-way ANOVA test. (S-V) Immunofluorescence(S) and quantification of relative F4/80⁺ macrophages (T), CX3CR1⁺ microglia (U), and OLFML3⁺ cells (V) in tumors from mice implanted with control and LOX-overexpressed GL261 cells and treated with clodronate liposomes (200 mL, once every 3 days) starting at day 4. Scale bar, 50 μm. n=3 independent samples. Student's t test. (W and X) Representative images (W) and quantification (X) of relative migration of THP-1 macrophages following stimulation with the conditioned media (CM) from U87 cells pretreated with or without SR9009 (5 μM). Scale bar, 200 μm. n=3 independent samples. Student's t test. (Y and Z) The concentration of SR9009 in the plasma (left; Y) and brain (right; Y) of mice after drug administration at indicated time points. n.s., not significant, P>0.05, *, P<0.05, **, P<0.01, ***, P<0.001.

FIGS. 10A-G. Effects of BAPN and SR9009 on Proliferation and Colony Formation in U87, PTEN-KO SF763, and 005 GSC Cells. (A and B) Representative images (A) and quantification (B) of proliferation of U87 cells treated with or without BAPN (200 μM) or SR9009 (5 μM). n=4 independent samples. One-way ANOVA test. (C and D) Representative images (C) and quantification (D) of proliferation of 005 GSCs treated with or without BAPN (200 μM) or SR9009 (5 μM). n=3 independent samples. One-way ANOVA test. (E-G) Representative images (E) and quantification (F and G) of colony formation of U87 and PTEN-KO SF763 cells treated with or without BAPN (200 μM) or SR9009 (5 μM). n=3 independent samples. One-way ANOVA test. n.s., not significant, P>0.05, *, P<0.05, **, P<0.01, ***, P<0.001.

FIGS. 11A-K. LOX regulates the NF-kB-PATZ1 signaling axis in PTEN-deficient GBM cells. (A) GSEA shows enrichment of RELA_DN.v1_DN signature in LOX shRNA (shLOX) versus shRNA control (shC) U87 cells. (B) Relative mRNA expression of 10 transcription factors (TFs) in U87 cells expressing shC and shLOX. n=3 independent samples. One-way ANOVA test. (C) Relative mRNA expression of 10 TFs in U251 cells treated with or without LOX inhibitor BAPN (200 μM). n=3 independent samples. Student's t test. (D) Relationship between PATZ1 and LOX expression in TCGA GBM patient tumors. Pearson test. (E) Relationship between Paired Related Homeobox 1 (PRRX1) and LOX expression in TCGA GBM patient tumors. Pearson test. (F) Immunoblots for P-P65 and P65 in lysates of GSC23 and GSC7-10 expressing shC and shLOX. (G) Immunoblots for PATZ1 in lysates of GSC23 and GSC7-10 expressing shC and shLOX. (H) Immunoblots for PATZ1 in lysates of PTEN-KO SF763 cells in the presence or absence of PATZ1 overexpression (OE). (I) Immunoblots for PATZ1 in lysates of PTEN-WT SF763 cells expressing shC and PATZ1 shRNA (shPATZ1). (J) Immunoblots for OLFML3 in lysates of U87 and PTEN-KO SF763 cells treated with or without SR9009 (5 μM) or BAPN (200 μM). (K) Immunoblots for OLFML3 in lysates of U87 and PTEN-KO SF763 cells treated with or without SR9009 (5 μM) or P65 inhibitor (P65i) SC75741 (5 μM). n.s., not significant, P>0.05, *, P<0.05, **, P<0.01, ***, P<0.001.

FIGS. 12A-D. Radiation upregulates CLOCK, OLFML3 and LOX in PTEN-deficient GBM cells. Immunoblots for CLOCK, OLFML3, and LOX in lysates of U251 cells (A), PTEN-KO LN229 cells (B), and 005GSC cells (C) treated with radiation at indicated doses. (D) Schematic diagram for the establishment and treatment in GBM tumor-bearing mice.

### DETAILED DESCRIPTION

The present disclosure provides compositions and methods for the treatment of cancer (e.g., glioblastoma). In particular, the present disclosure provides a dual targeting anti-tumor composition comprising a LOX inhibitor and a CLOCK-OLFML3 inhibitor, and methods of use thereof for treating glioblastoma in combination with or without an anti-programmed death-1 (PD1) therapy or radiotherapy.

In experiments conducted during development of embodiments herein, mechanisms underlying macrophages in suppression of microglia infiltration in the GBM TME were uncovered, which supports a therapeutic strategy that involves dual targeting macrophages and microglia, alone or in combination with either immunotherapy or radiotherapy. Experiments demonstrated that LOX inhibition in PTEN-deficient GBM upregulated OLFML3 to induce a compensatory increase of microglia infiltration into the GBM TME. Dual inhibition of LOX and CLOCK-OLFML3 axis extended the survival of GBM-bearing mice and lead to disease eradication in majority of tumor-bearing mice when combined with anti-PD1 therapy or radiotherapy.

A key feature of PTEN gene, is that it acts as a tumor suppressor and was originally isolated from a homozygous deletion on chromosome 10q23 of human GBM. Moreover, PTEN mutations/depletions are observed in about 30-40% of GBMs, which results in PI3K/AKT pathway activation, contributing to tumor progression and radiotherapy resistance. In addition to these cell intrinsic effects, findings revealed that PTEN loss contributes to generate an immunosuppressive GBM TME through a variety of mechanisms. For example, PTEN loss in GBM cells leads to immune escape via inducing T cell apoptosis and upregulating PD-L1 expression. Moreover, PTEN deletion in GBM cells results in upregulation of LOX, which, in turn, triggers the infiltration of macrophages into the GBM TME.

Further evidence was provided by evaluating PTEN-deficient GBM mouse models which showed that reducing macrophage infiltration via LOX inhibition enhances anti-tumor T cell immunity and synergizes with anti-PD1 therapy. These in vivo results coupled with the findings observed in GBM patients showing that PTEN mutations and macrophage abundance are enriched in anti-PD1 therapy nonresponders compared to responders, reinforces the importance of macrophages in regulating anti-PD1 therapy resistance and supports the treatment strategy of combining LOX inhibitors and ant-PD1 therapy specifically in PTEN-deficient GBM.

One of the key hallmarks of GBM is the robust infiltration of TAMs. PTEN-LOX and CLOCK-OLFML3 axes are key factors responsible for the infiltration of macrophages and microglia, respectively. The scRNA-Seq analysis in GBM patient tumors showed that macrophages compete with microglia for space in the GBM TME, where macrophages are negatively correlated with microglia. In exploring the molecular mechanism underlying this competition, it was observed that macrophage chemokine LOX negatively regulates the expression of microglia chemokine OLFML3 in GBM cells by regulating the NF-κB-PATZ1 signaling axis. In vivo, suppressing macrophage infiltration via LOX inhibition induces a compensatory increase of microglia, consistent with findings observed in Ccr2-KO GBM tumors. Experiments were conducted during development of embodiments herein to develop a combination therapy of suppressing the infiltration of both macrophages and microglia via inhibition of LOX and CLOCK-OLFML3 axis in PTEN-deficient GBM mouse models. Efforts have centered on developing CSF1R inhibitors to deplete TAMs in GBM, but the results showed that CSF1R inhibition only induces a transient anti-tumor effect caused by the compensatory changes in macrophages after the treatment in brain. Combined anti-CSF1R and anti-PD1 therapies in GBM mouse models shows a modest effect to extend survival. Consistent with these preclinical findings, a clinical trial with CSF1R inhibitor showed a minimal anti-tumor effect in recurrent GBM. These findings highlight that dual targeting macrophage and microglia infiltration using BAPN and SR9009 coupled with anti-PD1 therapy produces robust anti-tumor effect in PTEN-deficient GBM models.

Despite the extensive treatments, about 70% of GBM patients experience disease progression within one year. Radiotherapy could increase TAM infiltration through different mechanisms. For instance, radiotherapy can disrupt the tumor vasculature systems resulting in hypoxia, which, in turn, upregulates hypoxia-inducible factor 1 to promote macrophage infiltration into the TME. In exploring the specific molecular mechanisms underlying radiation-induced macrophage and microglia infiltration in PTEN-deficient GBM, it was observed that the expression of LOX, CLOCK and OLFML3 is upregulated in PTEN-deficient GBM cells upon radiation.

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise.

The present disclosure also contemplates other embodiments “comprising,” “consisting of” and “consisting essentially of,” the embodiments or elements presented herein, whether explicitly set forth or not.

As used herein, the term “effective amount” refers to the amount of a composition (e.g., pharmaceutical composition) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., pharmaceutical compositions of the present invention) to a subject or in vivo, in vitro, or ex vivo cells, tissues, and organs. Exemplary routes of administration to the human body can be through the eyes (e.g., intraocularly, intravitreally, periocularly, ophthalmic, etc.), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the terms “simultaneous”, “combination”, “simultaneously” and variants thereof refers to the administration of at least two agent(s) or therapies to a subject. In some embodiments, the co-administration of two or more agents or therapies is concurrent (e.g., in the same or separate formulations). As used herein, the active agents may be administered as separate dosage forms that are administered alternately or sequentially on the same or separate days. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. The single dosage form may include additional active agents for the treatment of the disease state. Thus, co-administration is especially desirable in embodiments where the co-administration of the agents or therapies lowers the requisite dosage of a potentially harmful (e.g., toxic) agent(s).

As used herein, the term “cancer(s)” refers to the physiological condition in mammals that is typically characterized by unregulated cell growth. Included in this definition are benign and malignant cancers as well as dormant tumors or micrometastatses. In some embodiments, the type of cancers includes but is not limited to, carcinoma, lymphoma, blastoma, sarcoma, and leukemia. In other embodiments, such cancers include but are not limited to, glioblastoma (GBM), including, e.g., proneural GBM, neural GBM, classical GBM and mesenchymal GBM. Other cancers include, for example, breast cancer, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, ovarian cancer, cervical cancer, liver cancer, bladder cancer, hepatoma, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

As used herein, the term “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

As used herein, the terms “treatment,” “treating,” and the like refer to obtaining a desired pharmacologic and/or physiologic effect against a particular disease, disorder, or condition. Preferably, the effect is therapeutic, i.e., the effect partially or completely cures the disease and/or adverse symptom attributable to the disease. The terms “treatment” and “treating” refers to reversing, alleviating, slowing down, inhibiting the progression of, preventing, or reducing the likelihood of the disease, disorder, or condition to which such term applies, or one or more symptoms or manifestations of such disease, disorder, or condition. In some embodiments, for example, the terms “treatment” and “treating” of a disease also includes providing relief from the symptoms or side-effects of the disease (including palliative treatment).

As used herein, the term “preventing” refers to prophylactic steps taken to reduce the likelihood of a subject (e.g., an at-risk subject) from contracting or suffering from a particular disease, disorder, or condition. The likelihood of the disease, disorder, or condition occurring in the subject need not be reduced to zero for the preventing to occur; rather, if the steps reduce the risk of a disease, disorder, or condition across a population, then the steps prevent the disease, disorder, or condition for an individual subject within the scope and meaning herein. In some embodiments, preventing refers to causing a disease, disorder, condition, or symptom or manifestation of such, or worsening of the severity of such, not to occur.

Provided herein are compositions and methods for the treatment of cancer (e.g., glioblastoma) therewith. In particular, the present disclosure provides compositions and methods of a dual targeting anti-tumor composition, a LOX inhibitor and a CLOCK-OLFML3 inhibitor, for treating glioblastoma in combination with or without an anti-programmed death-1 (PD1) therapy or radiotherapy.

Embodiments of the present disclosure include a dual targeting anti-tumor composition comprising a lysyl oxidase (LOX) inhibitor and a CLOCK-OLFML3 inhibitor.

In some embodiments, the LOX inhibitor is a pharmaceutical or chemical compound. In some embodiments, the LOX inhibitor inhibits the activity of LOX enzymes. In some embodiments, the LOX inhibitor suppresses macrophage infiltration and upregulates microglial infiltration. In some embodiments, the LOX inhibitor upregulates expression of Programmed Death-Ligand 1 (PD-L1) and OLFML3. In some embodiments, the LOX inhibitor modulates OLFML3 expression via the NF-kB-PATZ1 signaling axis. In some embodiments, the LOX inhibitor is an organic nitrile, such as β-aminopropionitrile (BAPN). In some embodiments, the LOX inhibitor is a BAPN derivative, including but not limited to, D,L-β-aminopropionitrile (D,L-BAPN), D-β-aminopropionitrile (D-BAPN), L-β-aminopropionitrile (L-BAPN), β-aminopropionitrile hydrochloride, β-aminopropionitrile phosphate salt, ester derivatives of BAPN, etc. In some embodiments, the LOX inhibitor is an aminomethylenethiophene (AMT), such as those described in Leung et al. J. Med. Chem. 2019, 62, 12, 5863-5884; incorporated by reference in its entirety. In some embodiments, the LOX inhibitor is a pan-lysyl oxidase inhibitor PXS-5505, such as those described in Chitty et al. Nat Cancer. 2023, 4, 1326-1344; incorporated by reference in its entirety. In some embodiments, the LOX inhibitor is a LOX-specific antibody (α-LOX), such as those described in Cox et al. Cancer Res. 2013, 73(6):1721-32; incorporated by reference in its entirety.

In some embodiments, the CLOCK-OLFML3 inhibitor is a chemical compound or a drug. In some embodiments, the CLOCK-OLFML3 inhibitor suppresses microglial infiltration. In some embodiments, the CLOCK-OLFML3 inhibitor suppresses OLFML3 expression. In some embodiments, the CLOCK-OLFML3 inhibitor is a REV-ERBα agonist, such as SR9009. For example, in some embodiments, CLOCK-OLFML3 inhibitors include, but are not limited to, SR9011, CRY1 agonists, CRY2 agonists, SHP656 and SHP1703 agonists. In some embodiments, the CLOCK-OLFML3 inhibitor is OLFML3 neutralizing antibody. For example, in some embodiments, the neutralizing antibody include, but are not limited to, rec9F8 and rec46A9.

In some embodiments, a LOX inhibitor and/or CLOCK-OLFML3 inhibitor is an inhibitor of LOX and/or CLOCK-OLFML3 activity. However, in other embodiments LOX and/or CLOCK-OLFML3 are inhibited by inhibiting expression of LOX and/or CLOCK-OLFML3 and/or inducing expression of a less active variant of LOX and/or CLOCK-OLFML3. In some embodiments, provided herein are inhibitors of LOX and/or CLOCK-OLFML3 expression. In some embodiments, an inhibitor is a small molecule, peptide, antibody, antibody fragment, a genome editing agent, etc. In particular embodiments, a LOX and/or CLOCK-OLFML3 inhibitor is a nucleic acid-based inhibitor. In some embodiments, the inhibitor is a small molecule, an aptamer, a siRNA, a shRNA, a miRNA, a morpholino, a ribozyme, an antisense nucleic acid molecule, a CRISPR-Cas9-based construct, a CRISPR-Cpf1-based construct, a meganuclease, a zinc finger nuclease, a transcription activator-like (TAL) effector (TALE) nuclease, etc.

In some embodiments, the LOX and/or CLOCK-OLFML3 inhibitor is a small interfering RNA (siRNA), also known as short interfering RNA or silencing RNA. In some embodiments, an siRNA is an 18 to 30 nucleotide, preferably 19 to 25 nucleotide, most preferred 21 to 23 nucleotide or even more preferably 21 nucleotide-long double-stranded RNA molecule. siRNA is involved in the RNA interference (RNAi) pathway where the siRNA interferes with the expression of a specific gene (e.g., LOX and/or CLOCK-OLFML3). siRNAs naturally found in nature have a well-defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest (e.g., LOX and/or CLOCK-OLFML3). Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, in some embodiments at least one RNA strand has a 5′-and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA and inhibit LOX and/or CLOCK-OLFML3 is envisioned in the present invention. In some embodiments, siRNA duplexes are provided composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair. 2′-deoxynucleotides in the 3′ overhangs are as efficient as ribonucleotides but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP). In some embodiments, provided herein are siRNA molecules that target and inhibit the expression (e.g., knock down) of the LOX and/or CLOCK-OLFML3.

A short hairpin RNA (shRNA) is a sequence of RNA that makes a tight hairpin turn that can be used to silence gene expression (e.g., of LOX and/or CLOCK-OLFML3) via RNA interference. In some embodiments, shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). The RISC binds to and cleaves RNAs which match the siRNA that is bound to (e.g., comprising the sequence of the LOX and/or CLOCK-OLFML3). In some embodiments, si/shRNAs to be used in the present invention are chemically synthesized using appropriately protected ribonucleoside phosphoramidites and a conventional DNA/RNA synthesizer. In some embodiments, provided herein are shRNA molecules that target and inhibit the expression (e.g., knock down) of LOX and/or CLOCK-OLFML3.

Further molecules effecting RNAi (and useful herein for the inhibition of expression of LOX and/or CLOCK-OLFML3) include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of the LOX and/or CLOCK-OLFML3 after introduction into target cells. In some embodiments, provided herein are miRNA molecules that target and inhibit the expression (e.g., knock down) of LOX and/or CLOCK-OLFML3.

Morpholinos (or morpholino oligonucleotides) are synthetic nucleic acid molecules having a length of about 20 to 30 nucleotides and, typically about 25 nucleotides. Morpholinos bind to complementary sequences of target transcripts (e.g., LOX and/or CLOCK-OLFML3) by standard nucleic acid base-pairing. They have standard nucleic acid bases which are bound to morpholine rings instead of deoxyribose rings and linked through phosphorodiamidate groups instead of phosphates. Due to replacement of anionic phosphates into the uncharged phosphorodiamidate groups, ionization in the usual physiological pH range is prevented, so that morpholinos in organisms or cells are uncharged molecules. The entire backbone of a morpholino is made from these modified subunits. Unlike inhibitory small RNA molecules, morpholinos do not degrade their target RNA molecules. Rather, they sterically block binding to a target sequence within a RNA and prevent access by molecules that might otherwise interact with the RNA. In some embodiments, provided herein are morpholino oligonucleotides that target and inhibit the expression (e.g., knock down) of LOX and/or CLOCK-OLFML3.

A ribozyme (ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage is well established. Since it was shown that hammerhead structures can be integrated into heterologous RNA sequences and that ribozyme activity can thereby be transferred to these molecules, catalytic antisense sequences can be engineered for almost any target sequence can be created, provided the target sequence contains a potential matching cleavage site. The basic principle of constructing hammerhead ribozymes is as follows: A region of interest of the RNA (e.g., a portion of LOX and/or CLOCK-OLFML3), which contains the GUC (or CUC) triplet, is selected. Two oligonucleotide strands, each usually with 6 to 8 nucleotides, are taken and the catalytic hammerhead sequence is inserted between them. In some embodiments, provided herein are ribozyme inhibitors oligonucleotides of LOX and/or CLOCK-OLFML3.

In some embodiments, LOX and/or CLOCK-OLFML3 is inhibited (and/or LOX and/or CLOCK-OLFML3 activity is inhibited) by modifying the LOX and/or CLOCK-OLFML3 sequence in target cells. In some embodiments, the alteration of LOX and/or CLOCK-OLFML3 is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes. In some aspects, a Cas nuclease and gRNA (including a fusion of crRNA specific for the target sequence (e.g., a sequence within LOX and/or CLOCK-OLFML3) and fixed tracrRNA) are introduced into the cell. In general, target sites at the 5′ end of the gRNA target the Cas nuclease to the target site, e.g., LOX and/or CLOCK-OLFML3, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence (e.g., sequence within LOX and/or CLOCK-OLFML3). In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. The CRISPR system can induce double stranded breaks (DSBs) at the SRC-3 target site, followed by disruptions or alterations as discussed herein. In other embodiments, Cas9 variants, deemed “nickases,” are used to nick a single strand at the target site (e.g., within LOX and/or CLOCK-OLFML3). Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression (e.g., to inhibit expression of LOX and/or CLOCK-OLFML3). In some embodiments, the CRISPR system is used to alter LOX and/or CLOCK-OLFML3, inhibit expression of LOX and/or CLOCK-OLFML3, and/or to inactivate the expression product of the LOX and/or CLOCK-OLFML3.

The term “antisense nucleic acid molecule” or “antisense oligonucleotide” as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901). In some embodiments, provided herein are antisense oligonucleotides capable of inhibiting expression of LOX and/or CLOCK-OLFML3 when administered to cell or subject. In some embodiments, the antisense oligonucleotides are antisense DNA-and/or RNA-oligonucleotides. In some embodiments, provided herein are modified antisense oligonucleotides, such as, antisense 2′-O-methyl oligo-ribonucleotides, antisense oligonucleotides containing phosphorothiaote linkages, antisense oligonucleotides containing Locked Nucleic Acid LNA (R) bases, morpholino antisense oligonucleotides, PPAR-gamma agonists, antagomirs. In some embodiments, ASOs comprise Locked Nucleic Acid (LNA) or 2′-methoxyethyl (MOE) modifications (internucleotide linkages are phosphorothioates interspersed with phosphodiesters, and all cytosine residues are 5′-methylcytosines).

In some embodiments, the LOX inhibitor and the CLOCK-OLFML3 inhibitor are administered simultaneously or independently.

Further provided herein are methods for treating cancer in a subject comprising administering a dual targeting anti-tumor composition

In some embodiments, provided herein are methods for treating cancer in a subject comprising co-administering an effective amount of the dual targeting anti-tumor composition with an effective amount of a cancer therapeutic to a subject in need of treatment thereof.

In some embodiments, methods herein further comprise administering the dual targeting anti-tumor composition and the cancer therapeutic simultaneously or independently.

In some embodiments, the cancer is brain cancer. In some embodiments, the cancer is glioblastoma (GBM).

In some embodiments, the GBM is PTEN-deficient.

In some embodiments, the cancer therapeutic is an immune checkpoint inhibitor. Immune checkpoint inhibitors are a class of drugs designed to enhance the body's natural immune response against cancer and other diseases by blocking certain molecules (checkpoints) that regulate immune responses. Immune checkpoint molecules are ac component of systems that helps maintain immune balance and prevent excessive immune activation. Cancers often exploit immune checkpoint pathways to evade detection and attack by the immune system. By inhibiting specific immune checkpoints, immune checkpoint inhibitors can reactivate the immune response, enabling immune cells to recognize and attack cancer cells more effectively. Immune checkpoint inhibitors work by targeting specific cell surface molecules on immune cells or cancer cells. These molecules are often ligands or receptors that, when engaged, suppress immune responses. By blocking the interactions between these molecules, checkpoint inhibitors can enhance immune responses against cancers.

In some embodiments, the cancer therapeutic is an Anti-Programmed Death 1 (PD1) therapy. For example, in some embodiments, PD1 therapies include but are not limited to, Pembrolizumab (Keytruda), Nivolumab (Opdivo), Cemiplimab (Libtayo), Prolgolimab (Biocad), and Ivonescimab (Akeso). In some embodiments, the PD1 therapy is a small molecule inhibitors. For example, in some embodiments, the small molecule inhibitor includes, but is not limited to JBI-2174, etc. In some embodiments, the cancer therapeutic is a PD-L1 inhibitor, such as, Atezolizumab (Tecentriq), Avelumab (Bavencio), Durvalumab (Imfinzi), etc.

In some embodiments, the cancer therapeutic is a radiotherapy. For example, in some embodiments, radiotherapies include but are not limited to, Conventional External Beam Radiation Therapy (EBRT), Intensity-Modulated Radiation Therapy (IMRT), Image-guided radiation therapy (IGRT), Tomotherapy, Stereotactic body Radiation Therapy, Stereotactic Radiosurgery (SRS), Proton Beam Therapy, Brachytherapy, Intraoperative Radiation Therapy (IORT) and any variations thereof.

In some embodiments, the subject is a human.

In some embodiments, the manufacture of a medicament for the treatment of cancer in a subject in need thereof.

### Experimental

The following Materials/Methods and Examples are offered as illustrative as a partial scope and particular embodiments of the disclosure and are not meant to be limiting of the scope of the disclosure.

The present disclosure describes tumor-associated macrophages and microglia (TAMs) that are critical for tumor progression and therapy resistance in glioblastoma (GBM), a type of incurable brain cancer. Lysyl oxidase (LOX) and olfactomedin like-3 (OLFML3) have been shown to be essential macrophage and microglia chemokines in PTEN-deficient and CLOCK-overexpressing GBM, respectively. Single-cell transcriptomics and multiplex sequential immunofluorescence was used followed by functional studies to demonstrate that macrophages suppress microglia infiltration in GBM. LOX inhibition in PTEN-deficient GBM cells upregulates OLFML3 expression via the NF-kB-PATZ1 signaling pathway, inducing a compensatory increase of microglia infiltration. Dual targeting macrophages and microglia via inhibition of LOX and the CLOCK-OLFML3 axis generates potent anti-tumor effects that are further heightened for a complete regression in more than 60% of subjects when combined with anti-PD1 therapy or radiotherapy in PTEN-deficient GBM mouse models. Together these findings provide a translational triple therapeutic strategy for this lethal disease.

### Materials/Methods

Cell Culture. The GBM cell lines U87, U251, SF763, LN229, and CT2A, as well as 293T cells were cultured in Dulbecco's Modified Eagle's Medium (DMEM; Gibco, #11995-065). The mouse glioma cell line GL261 cells and SIM-A9 microglia were cultured in Dulbecco's Modified Eagle Medium-Ham's F12 medium (Gibco, #10565-018). HMC3 microglia were cultured in Eagle's Minimum Essential Medium (ATCC, #30-2003). THP-1 macrophages were cultured in RPMI 1640 medium (Gibco, #11875093). All cell lines were cultured in the indicated medium containing 10% fetal bovine serum (FBS; Fisher Scientific, #16140071) and 1:100 antibiotic-antimycotic (Gibco, #15140-122), and were purchased from the American Type Culture Collection (ATCC). Human GSCs (GSC23 and GSC7-10) and mouse GBM tumor-derived 005 GSCs and QPP7 GSCs were cultured in neural stem cell (NSC) proliferation media (Millipore, #SCM005) containing 20 ng/mL epidermal growth factor (EGF; PeproTech, #AF-100-15) and basic fibroblast growth factor (bFGF; PeproTech, #100-18B). Human GSCs were gifted by Dr. Frederick F. Lang from the Brain Tumor Center (The University of Texas MD Anderson Cancer Center). 005 GSCs and QPP7 GSCs were provided by Dr. Samuel D. Rabkin (Massachusetts General Hospital) and Dr. Jian Hu (The University of Texas MD Anderson Cancer Center), respectively. PTEN CRISPR KO in SF763 and LN229 cells were generated. All cells were confirmed to be mycoplasma-free and were maintained at 37° C. and 5% CO₂. Cells were treated with BAPN (Sigma-Aldrich, #B-A3134, 200 μM), SR9009 (Cayman, #11929, 5 μM), SC75741 (MedChem Express, #HY-10496, 5 μM), and/or NF-kB activator 1 (MedChemExpress, #HY-134476, 1 μM) for 24 hours for protein expression analysis or 8 hours for mRNA expression analysis.

Mice and Intracranial Xenograft Tumor Models. Female C57BL/6 mice at 3 to 4 weeks of age were purchased from the Jackson Laboratory (#0000664). All animals were grouped by 5 mice per cage and maintained in IVC System (San Diego, CA) for a week before the experiment. All animal experiments were performed with the approval of the Institutional Animal Care and Use Committee (IACUC) at Northwestern University. The intracranial xenograft tumor models were established previously. In brief, mice were anesthetized by isoflurane through IMPAC6 Anesthesia System. Then a dental drill was used to open a small hole in the skull of mice 1.2 mm anterior and 3.0 mm lateral to the bregma. Mice were placed into the stereotactic apparatus, and 5 μL 005 GSC, CT2A, or GL261 cells in FBS-free culture medium were injected into the right caudate nucleus 3 mm below the surface of the brain using a 10 μL Hamilton syringe with an unbeveled 30-gauge needle. The incision was closed using Vetbond glue. Meloxicam (20 mg/kg, daily) was subcutaneously injected for pain relief for 3 days after surgery. Mice received the treatment with BAPN (2 g/L in drinking water) on day 4, SR9009 (100 mg/kg body weight, i.p.) daily for 10 days beginning at day 7 post-orthotopic injection, anti-PD1 (10 mg/kg body weight, i.p.) on days 11, 14, and 17 post-orthotopic injection, and/or clodronate liposomes (200 μL, once every 3 days) starting at day 4 post-orthotopic injection. Mice with neurological deficits or moribund appearance were sacrificed according to the IACUC protocol. At the end of the experiment, the brains of mice were collected, either fixed in 4% paraformaldehyde (PFA) (ThermoFisher Scientific, #J61899.AK) after transcardiac perfusion with PBS for optimal cutting temperature (OCT)-cryosectioning or processed using the percoll density gradient cell separation method to isolate tumor-derived immune cells for flow cytometry analysis.

Mass spectrometry. A high-performance liquid chromatography tandem mass spectrometry (LC-MS/MS) assay was developed to quantify BAPN and SR9009 in plasma and brain of C57BL/6 mice. Specifically, the blood and brain tissues were collected after 1, 2, 4, and 8 hours of the administration of BAPN (2 g/L in drinking water) or SR9009 (100 mg/kg body weight, i.p.). The plasma was generated using the standard centrifugation techniques, and the brain tissues were pulverized by cryogenic grinding with liquid nitrogen. The plasma and brain tissue samples were mixed with internal standards, deproteinized with MeOH, and processed into LC-MS/MS to test the concentration of BAPN or SR9009. The analysis was performed at the Mass Spectrometry Core in Research Resources Center of University of Illinois at Chicago.

Computational analysis of human GBM datasets. For analysis of human GBM data, the gene expression data of TCGA datasets (Agilent-4502A and/or HG-U133A microarrays) from GlioVis was used. The expression, correlation, and GSEA of interesting genes and gene signatures in GBM patients were performed.

GSEA analysis. GSEA software 4.1.0 from the Broad Institute was used. The gene expression data from microarray data of public available GEO and newly generated RNA sequencing data of U87 cells were used for performing GSEA. The gene Ontology Biological Process (GOBP) signatures were downloaded from the Molecular Signatures Database. The normalized enrichment score (NES) and false discovery rate (FDR) were acquired by the analysis, with FDR<0.25 was considered statistically significant.

Single-cell sequencing data analysis. The scRNA-seq data of GEO, GSE131928, were used to analyze expression pattern of LOX in glioma cells and the distribution of myeloid cells, including macrophages, monocytes, microglia, and DCs, in tumors of glioma patients (LGG, ndGBM and rGBM). Based on their abundance in GBM tumors, the correlation between macrophages and microglia was analyzed.

Plasmids and viral transfections. Short hairpin RNA (shRNA) targeting human LOX and PATZ1 and mouse Clock in the pLKO.1 vector (Sigma, #SHC001) were used. Lentiviral particles were generated and used. In brief, 8 μg of the shRNA plasmid, 4 μg of the psPAX2 plasmid (Addgene, #12260), and 2 μg of the pMD2.G plasmid (Addgene, #12259) were transfected into 293T cells plated in 100-mm dishes using Lipofectamine 2000 (Invitrogen, #13778150). Supernatant with lentiviral particles was collected and filtered at 48 and 72 hours after transfection. Cells were infected with viral supernatant containing 10 μg/mL polybrene (Millipore, #TR-1003-G). After 48 hours, cells were selected by puromycin (10 μg/mL; Millipore, #540411) and tested for the expression of LOX, PATZ1, and CLOCK by immunoblots. The following human and mouse shRNA sequences (LOX: #3: TRCN0000286463 and #4: TRCN0000286532; PATZ1: #4: TRCN0000274379 and #5: TRCN0000274416; and Clock: #1: TRCN0000095686 and #2: TRCN0000306474) were selected for further use following the validation.

For gene overexpression, plasmids of human Tagged Lenti ORF Clone of PATZ1 (Origene, #RC211869L4) and mouse Tagged Lenti ORF Clone of Lox (Origene, #MR206463L4) were used. These plasmids were transformed into high-efficiency chemically competent Escherichia coli cells (ThermoFisher Scientific, #C737303) and recovered in Lysogenia broth (LB, Fisher BioReagents, #BP9723). Post-recovery, LB containing E. coli transformants were plated on LB selection plates containing 34 μg/mL chloramphenicol (Fisher BioReagents, #BP904) for 16 hours of incubation at 37° C. to select clones containing the gene expression vectors. The selected colonies were picked from the selection plates for inoculation in LB broth supplemented with 34 μg/mL chloramphenicol to maintain the selection, and then further purified for plasmid DNA using a QIAprep Spin Miniprep Kit (Qiagen, #27106). Purified plasmids were transfected into cells using lentiviral transfection methodology.

Immunofluorescence. Immunofluorescence was performed using a standard protocol that was previously established. In brief, slides from cryosections were kept at room temperature for 30 minutes and fixed in 10% PFA for 30 minutes prior to permeabilization. Then, 0.25% Triton X-100 in PBS was added for 30 minutes at room temperature to permeabilize the cell membrane. After three times PBS washing, sections were blocked by 5% goat serum for 30 minutes. Specimens were incubated with primary antibody for 1 hour at room temperature and then overnight at 4° C. Then, the unbound primary antibodies were washed out by three times PBS for 3 minutes each and corresponding secondary antibody cocktails were prepared and added to the sections for 1 hour incubation. Cell nuclear was counterstained with DAPI/anti-fade mounting medium (Vector Laboratories, #H-1200-10). Immunofluorescence images were captured using Nikon AX/AX R Confocal Microscope System with an apo 60 1.40 Oil 160/0.17 objective in the Center for Advanced Microscopy (CAM) at Northwestern University. The relative intensity of the protein signal was determined by Image J. For one slide, 3-5 fields of images were captured randomly, and the intensity of the protein signal was determined by Image J. The average quantified value of these 3-5 fields was represented as the protein signal intensity of one sample and presented as the individual point in the bar graph. The number of replicates for each experiment is indicated in the figure legends. Antibodies specific to CD8 (Invitrogen, #PA5-81344), CD69 (Santa Cruz Biotechnology, #sc-373799), CX3CR1 (Invitrogen, #702321), F4/80 (Cell Signaling Technology, #30325S), OLFML3 (Invitrogen, #702321), Ki67 (Cell Signaling Technology, #9129S), and CC3 (Cell Signaling Technology, #9661S) were used.

Sequential immunofluorescence (SeqIF™) multiplexing and microscopy. Automated multiplexed seqIF staining and imaging were performed on FFPE sections at Northwestern University using the COMET™ platform (Lunaphore Technologies). The multiplexed panel was comprised of 4 antibodies: GFAP (Abcam, #ab68428), CD31 (Abcam, #ab182981), P2RY12 (Atlas Antibodies, #HPA014518), and CD163 (Abcam, #ab182422). The 4-plex protocol was generated using the COMET™ Control Software, and reagents were loaded onto the COMET™ device to perform seqIF. All antibodies were validated using conventional IHC and/or IF staining in conjunction with corresponding fluorophores and DAPI (ThermoFisher Scientific, #D21490). For optimal concentration and best signal-to-noise ratio, all antibodies were tested at 3 different dilutions: starting with the manufacturer-recommended dilution (MRD), MRD/2, and MRD/4. Secondary Alexa fluorophore 555 (Invitrogen, #A32732) and Alexa fluorophore 647 (Invitrogen, #A32733) were used at 1:200 or 1:400 dilutions, respectively. The optimizations and full runs of the multiplexed panel were executed using the technology integrated in the Lunaphore COMET™ platform (characterization 2 and 3 protocols, and seqIF™ protocols, respectively). The seqIF™ workflow was parallelized on a maximum of 4 slides, with automated cycles of iterative staining of 2 antibodies at a time, followed by imaging, and elution of the primary and secondary antibodies, with no sample manipulation during the entire workflow. All reagents were diluted in Multistaining Buffer (BU06, Lunaphore Technologies). The elution step lasted 2 minutes for each cycle and was performed with Elution Buffer (BU07-L, Lunaphore Technologies) at 37° C. Quenching lasted for 30 seconds and was performed with Quenching Buffer (BU08-L, Lunaphore Technologies). Staining was performed with incubation times set at 4 and 2 minutes for primary antibodies and secondary antibodies, respectively. Imaging was performed with an integrated epifluorescent microscope at 20× magnification with Imaging Buffer (BU09, Lunaphore Technologies) and exposure times set for DAPI 80 ms, Cy5 200 ms, TRITC 400 ms. Image registration was performed immediately after concluding the staining and imaging procedures by COMET™ Control Software. Each seqIF™ protocol resulted in a multi-layer OME-TIFF file where the imaging outputs from each cycle were stitched and aligned. COMET™ OME-TIFF files contain a DAPI image, intrinsic tissue autofluorescence in TRITC and Cy5 channels, and a single fluorescent layer per marker. The intrinsic tissue autofluorescence signals were subtracted from the subsequent cycles and the markers were subsequently pseudocolored for visualization of multiplexed staining results in the Viewer from Lunaphore.

Hematoxylin and Eosin (H&E) staining. Staining was performed using the H&E staining kit (Abcam, #ab245880) according to a standard protocol. In brief, the FFPE sections were baked at 65° C. for 2 hours and then were subjected to xylene and ethanol for deparaffinization and rehydration. After that, the sections were incubated with hematoxylin, Mayer's (Lillie's Modification) for 5 minutes, and then incubated with the Bluing Reagent and Eosin Y Solution (Modified Alcoholic) for 15 seconds and 3 minutes, respectively. After washing, slides were dehydrated in three changes of absolute alcohol and the images of tissue sections were captured using TissueFAXS in the CAM at Northwestern University.

Quantitative real-time PCR (RT-qPCR). Cells were pelleted, and RNA was isolated with the RNeasy Mini Kit (Qiagen, #74106) from a protocol that was previously established. RNA was quantified by NanoDrop spectrophotometers and then the All-In-One 5× RT MasterMix (Applied Biological Materials, #G592) was used to reverse-transcribe RNA into cDNA in T100 Thermal Cycler (Bio-Rad). RT-qPCR was performed with the use of SYBR Green PCR Master Mix (Bio-Rad, #1725275) in CFX Connect Real-Time PCR Detection System (Bio-Rad). Primers used for RT-qPCR were listed in Table 1 below. The expression of each gene was normalized to that of housekeeping gene GAPDH.

TABLE 1
A list of primers used for RT-qPCR and ChIP-qPCR.

| Gene name | Forward | Reverse |
|---|---|---|
| OLFML3 | TCCTTTTGTCATGGTCGGGAC | TAAAGCAGCTAGTCGGCGTTC |
| | (SEQ ID NO: 1) | (SEQ ID NO: 2) |
| PATZI | GATGCACACTATCAGCTCCAAG | CGATAACCGACCTCATCAGCA |
| | (SEQ ID NO: 3) | (SEQ ID NO: 4) |
| MAFF | TGCCCAGGTCCCATTTCTC (SEQ | GGCCCACGAAGGGAATGT (SEQ |
| | ID NO: 5) | ID NO: 6) |
| RELB | CAGCCTCGTGGGGAAAGAC | GCCCAGGTTGTTAAAACTGTGC |
| | (SEQ ID NO: 7) | (SEQ ID NO: 8) |
| ETV1 | GGCCCCAGGCAGTTTTATGAT | GATCCTCGCCGTTGGTATGT (SEQ |
| | (SEQ ID NO: 9) | ID NO: 10) |
| SNAIL | TCGGAAGCCTAACTACAGCGA | AGATGAGCATTGGCAGCGAG |
| | (SEQ ID NO: 11) | (SEQ ID NO: 12) |
| ATF3 | CCTCTGCGCTGGAATCAGTC | TTCTTTCTCGTCGCCTCTTTTT |
| | (SEQ ID NO: 13) | (SEQ ID NO: 14) |
| KLF6 | GGCAACAGACCTGCCTAGAG | CTCCCGAGCCAGAATGATTTT |
| | (SEQ ID NO: 15) | (SEQ ID NO: 16) |
| TWIST1 | GTCCGCAGTCTTACGAGGAG | GCTTGAGGGTCTGAATCTTGCT |
| | (SEQ ID NO: 17) | (SEQ ID NO: 18) |
| PRRX1 | CAGGCGGATGAGAACGTGG | AAAAGCATCAGGATAGTGTGTCC |
| | (SEQ ID NO: 19) | (SEQ ID NO: 20) |
| JUNB | ACGACTCATACACAGCTACGG | GCTCGGTTTCAGGAGTTTGTAGT |
| | (SEQ ID NO: 21) | (SEQ ID NO: 22) |
| GAPDH | GGAGCGAGATCCCTCCAAAAT | GGCTGTTGTCATACTTCTCATGG |
| | (SEQ ID NO: 23) | (SEQ ID NO: 24) |
| OLFML3 | GAGGTGGTTAAGGCTTCTG | CTCTCACTACTCTGGAATGC (SEQ |
| #1-1 (ChIP) | (SEQ ID NO: 25) | ID NO: 26) |
| OLFML3 | GAGGTGGTTAAGGCTTCTG | CTCACTACTCTGGAATGCTT (SEQ |
| #1-2 (ChIP) | (SEQ ID NO: 27) | ID NO: 28) |
| OLFML3 | CCTGCTTGACTGCCTAGA (SEQ | AGCCAGAAGCCTTAACCA (SEQ |
| #2-1 (ChIP) | ID NO: 29) | ID NO: 30) |
| OLFML3 | CCTGCTTGACTGCCTAGA (SEQ | GCAGCCAGAAGCCTTAAC (SEQ |
| #2-2 (ChIP) | ID NO: 31) | ID NO: 32) |
| OLFML3 | GGTGAAGTCTAAGCCTTAGT | GGTCAAGCCCAACTTATTC (SEQ |
| #3-1 (ChIP) | (SEQ ID NO: 33) | ID NO: 34) |
| OLFML3 | GGTGAAGTCTAAGCCTTAGT | AAGGGAGGAAGCAGGAAA (SEQ |
| #3-2 (ChIP) | (SEQ ID NO: 35) | ID NO: 36) |

ChIP-PCR. ChIP-PCR was performed using the commercial Pierce™ Magnetic CHIP kit (ThermoFisher, #26157) from a protocol that was previously established. In brief, PTEN-KO SF763 cells were cross-linked with 1% PFA for 10 minutes, and then reactions were quenched using the glycine solution for 5 minutes at room temperature. Cells were then lysed with membrane extraction buffer for 10 minutes on ice, and the chromatin fragmentation was generated by Mnase digestion followed by sonication using three 20-second pulses at 3-watt power. After that, the solubilized chromatin was incubated with PATZ1 (Santa Cruz Biotechnology, #sc-393223 X) antibody overnight at 4° C. followed by 2 hours incubation with CHIP Grade Protein A/G Magnetic Beads with mixing. Immune complexes were then washed with IP Wash Buffer I three times and IP Wash Buffer II once. Elution Buffer was added to the sample for elution at 65° C. for 30 minutes. Then proteinase K (20 mg/mL) and NaCl (5M) were added for reverse-crosslinking at 65° C. for 1.5 hours. Eluted DNA was purified using DNA Clean-Up Column and then used to perform PCR. The OLFML3 primers were designed according to the E-box of the human OLFML3 gene and were listed in Table 1 above.

Immunoblotting. The protein expression of cells was tested by Western blotting analysis from a protocol that was previously established. In brief, cells were lysed on ice with RIPA lysis buffer (Thermo Scientific, #89900) supplemented with Protease Inhibitor Cocktail (Thermo Scientific, #78429). BCA Protein Assay Kit (Thermo Fisher Scientific, #PI23225) was used to measure protein concentration. Protein solution was mixed with the LDS sample buffer and heated at 95° C. for 10 minutes. After that, protein samples were loaded to SurePAGE gels (GenScript, #M00653) and then transferred to 0.2 μm nitrocellulose (NC) membrane (Bio-Rad, #1620112) using a preprogrammed standard protocol for 30 minutes in the Trans-Blot Turbo system (Bio-Rad). NC membranes were blocked using 5% dry milk in TBST for 1 hour at room temperature and then incubated with primary antibodies (1:1,000 dilution) overnight at 4° C. After washing three times, membranes were incubated with HRP-conjugated anti-mouse (Cell Signaling Technology, #7076S) or anti-rabbit (Cell Signaling Technology, #7074S) secondary antibodies for 1 hour at room temperature. After washing, membranes were incubated with ECL substrate and imaged under ChemiDoc Touch Imaging System (Bio-Rad). Antibodies were purchased from the indicated companies, which include β-actin (Cell Signaling Technology, #3700S), LOX (Abcam, #ab174316), CLOCK (Cell Signaling Technology, #5157S), OLFML3 (Abcam, #ab111712), PD-L1 (Cell Signaling Technology, #64988S), P-P65 (Cell Signaling Technology, #3033S), P65 (Cell Signaling Technology, #8242S), and PATZ1 (Santa Cruz, #sc-393223 X).

Migration assay. To study the relationship between macrophages and microglia, HMC3 microglia or THP-1 macrophages were seeded into the 24-well plates (receiver wells) using serum-containing culture medium for 24 hours to reach 40-60% confluence and then stimulated with or without U87 cell CM for additional 24 hours. After that, cells were washed with PBS twice and cultured in serum-free culture medium. For upper chambers, THP-1 macrophages (2×10⁵) and HMC3 microglia (5×10⁴) were suspended in serum-free culture medium and seeded into 5.0 mm (Corning, #3421) and 8.0 mm (Corning, #3422) 24-well Transwell inserts, respectively. To study the role of GBM cells on microglia migration, HMC3 microglia (5×10⁴) were suspended in serum-free culture medium and seeded into 8.0 μm inserts. SIM-A9 microglia (1×10⁵) and THP-1 macrophages (1×10⁵) were suspended in serum-free culture medium and seeded into 5.0 μm inserts. The CM from LOX-depleted or inhibited U87 cells, LOX-overexpressed GL261 cells, or SR9009-treated U87 cells were added to the receiver wells, respectively. After 10 hours, migrated cells were fixed with 4% PFA (ThermoFisher, #J61899.AK) for 30 minutes and stained with crystal violet (Sigma-Aldrich, #C-3886) for another 30 minutes. The membrane inserts were washed with water and imaged under an EVOS microscope. The number of transferred cells was counted using ImageJ.

Tumor-derived immune cells isolation. Mice with neurologic deficits or moribund appearance were sacrificed to harvest their brains. Immune cells in the brain tumors were isolated using the percoll density gradient cell separation method as previously described. In brief, after perfusion with PBS, brains were homogenized on ice with pre-cold 10 mL HBSS. Then cells were spun down at 1,500 rpm for 10 minutes at 4° C., and were resuspended in 30% Percoll (GE Healthcare, #17-0891-01). The solution was gently laid on top of the 70% Percoll and centrifuged at 1,200 g for 30 minutes at 4° C. with accelerator 7 and breaker 0. After removing myelin and debris, the interphase was collected and centrifuged at 1,500 rpm for 10 minutes at 4° C. The cell pellet was resuspended for further analysis.

Flow cytometry. The single-cell suspensions were incubated with fixable viability dye (Invitrogen, #5211229035) on ice for 10 minutes. After washing with FACS buffer, cells were incubated with the TruStain FcX (anti-mouse CD16/32) Antibody (BioLegend, #103132) and True-Stain Monocyte Blocker (BioLegend, #426102) in 5% BSA for 30 minutes on ice to block Fc receptors and non-specific binding of the cyanine acceptor fluorophores. Different antibody cocktails, including Percp/Cy5.5 anti-mouse CD45 (BioLegend, #103132), AF488 anti-mouse CD3 (BioLegend, #100210), BV711 anti-mouse CD8 (BioLegend, #100747), PE/Cy7 anti-mouse CD69 (BioLegend, #104512), APC/Cy7 anti-mouse IFN-γ (BioLegend, #505850), PE/Cy7 anti-mouse/human CD11b (BioLegend, #101216), PE anti-mouse CD68 (BD Bioscience, #566386), and BV421 anti-mouse CX3CR1 (BD Bioscience, #567531) were added to the samples and incubated for 30 minutes on ice. After washing with FACS buffer, cells were incubated with fixation buffer (BioLegend, #420801) overnight. Samples were read through the BD FACSymphony or BD LSRFortessa flow cytometer and analyzed in FlowJo v10.8.1.

Proliferation (CFSE) assay. Cell proliferation was assessed using the CellTrace carboxy fluorescein succinimidyl ester (CFSE) Cell Proliferation Kit (Invitrogen, #C34554). Briefly, 1×10⁶ cells were collected and incubated with CFSE working solution (1:1,000) for 20 minutes at 37° C. The staining was stopped by adding complete cell culture media. After washing, cells were cultured for 3 days with or without the treatment of BAPN (Sigma-Aldrich, #B-A3134, 200 μM), or SR9009 (Cayman, #11929, 5 μM) in the dark and used for flow cytometry analysis. The percentage of CFSE positive peaks over the undivided peak (generation 0) was analyzed using FlowJo v10.8.1.

Colony formation assay. 1×10³ GBM cells were seeded in each well of 6-well plates with or without the treatment of BAPN (Sigma-Aldrich, #B-A3134, 200 μM), or SR9009 (Cayman, #11929, 5 μM). After 7-10 days, cells were fixed and stained with 0.5% crystal violet in 25% methanol for 1 hour. After three times washing by PBS, the plates were scanned, and the colony number was counted using ImageJ.

Patient samples. Tumor samples from surgically resected IDH-WT GBMs were collected at Northwestern Memorial Hospital following the approved Institutional Review Board (IRB) protocol (STU00214485). Three ndGBM patients (#ITA-13, male, 62-year-old; #ITA-19, female, 50-year-old; and #ITA-26, female, 50-year-old) were diagnosed according to the WHO diagnostic criteria. Formalin fixed paraffin embedded blocks and slides were prepared and handled by the Northwestern Central Nervous System Tumor Bank.

Statistical analysis. Statistical analyses were performed with one-way ANOVA tests for comparisons among groups and Student t-tests for comparisons between two groups. Data were represented as mean±SD. Correlation analysis was conducted using the Pearson test to determine the Pearson correlation coefficient (R-value) and P-value. The survival analysis for animal models was determined by conducting Log-rank (Mantel-Cox) test. All statistical analyses were performed using GraphPad Prism 10 (GraphPad Software, USA). The P values were designated as *, P<0.05; **, P<0.01; ***, P<0.001; and n.s., not significant (P>0.05).

### Example 1

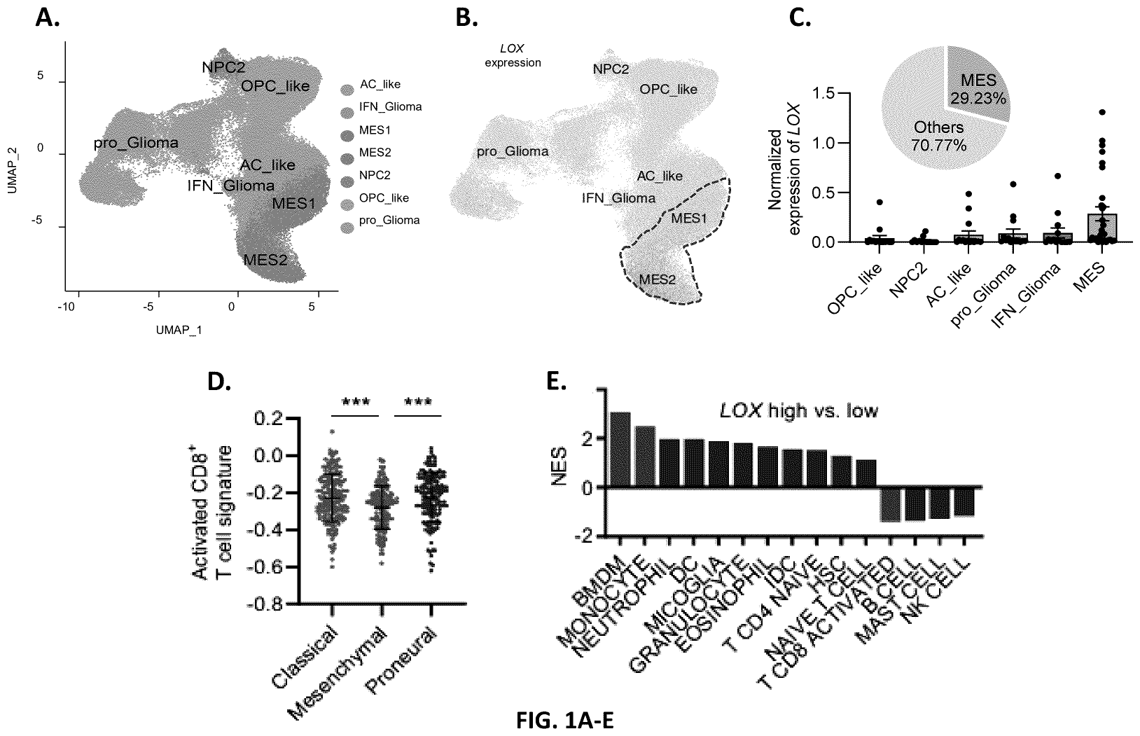

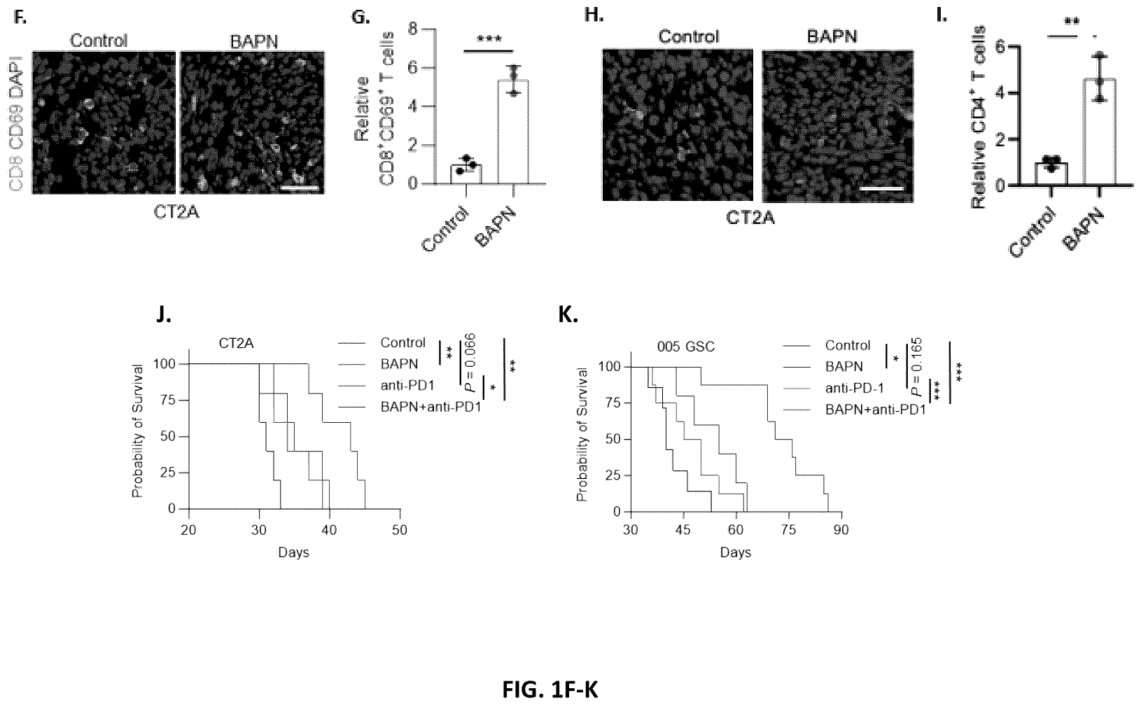

Targeting LOX improves the efficacy of anti-PD1 therapy in PTEN-deficient GBM. Findings have revealed that the macrophage chemokine LOX is upregulated in PTEN-deficient GBM cells. To confirm the expression pattern of LOX, single-cell RNA sequencing (scRNA-Seq) data was analyzed from GBM patient tumors with results showing that LOX was highly expressed in mesenchymal GBM cells, which account for 29.23% of total malignant cells (FIG. 1A-C). PTEN deficiency is common in mesenchymal GBM subtype, which harbors higher immunosuppressive macrophages relative to classical and proneural GBMs. Accordingly, bioinformatic analysis in The Cancer Genome Atlas (TCGA) GBM dataset demonstrated that the activated CD8⁺ T cell signature was reduced in mesenchymal tumors compared to classical and proneural tumors (FIG. 1D). To identify specific immune cells linked to LOX expression in GBM, TCGA GBM tumors were audited for 15 types of immune cells with validated gene set signatures. Bone marrow-derived macrophage (BMDM) and monocyte were identified as the top immune cell types enriched in LOX-high tumors compared to LOX-low tumors. Conversely, an activated CD8⁺ T cell signature was reduced in LOX-high tumors (FIG. 1E). These findings suggested there was a potential connection between LOX-regulated macrophages and T cells in GBM.

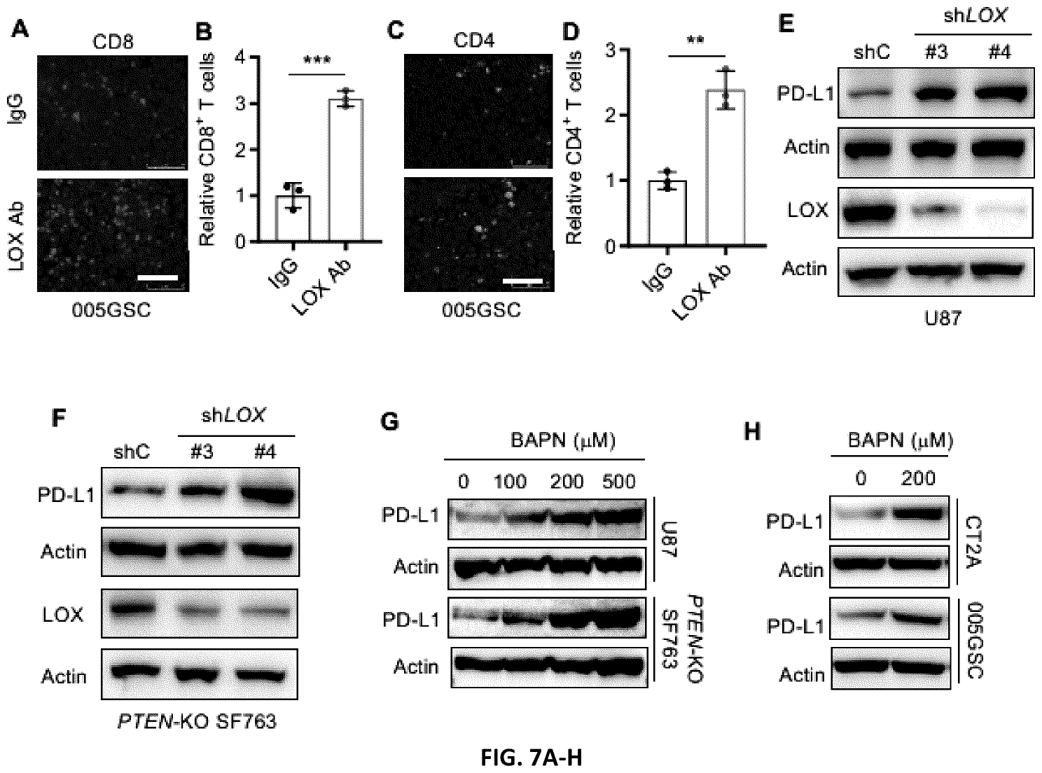

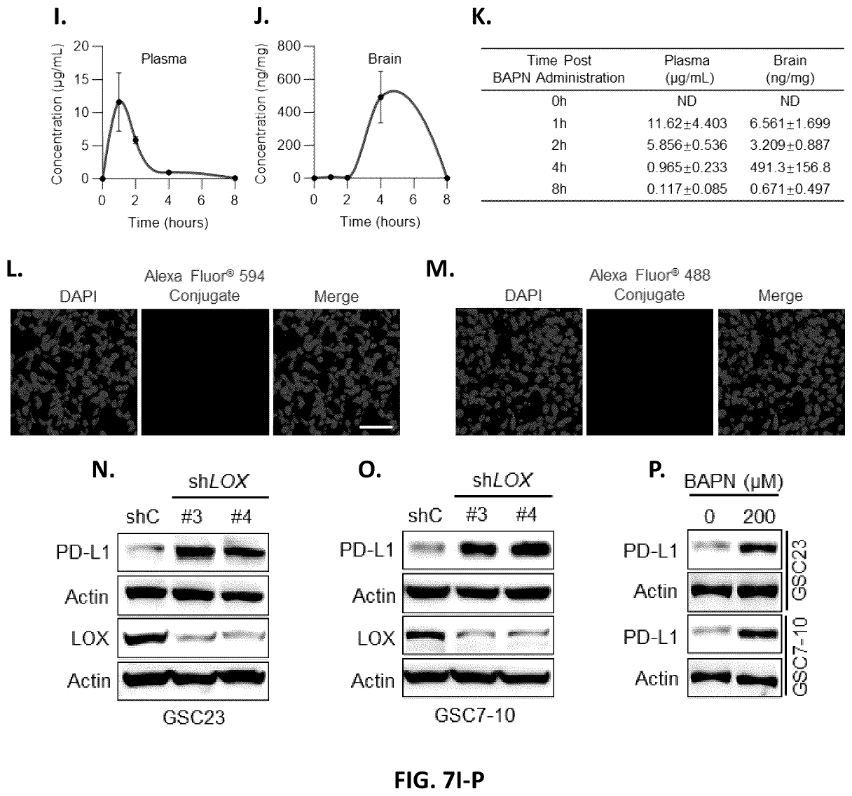

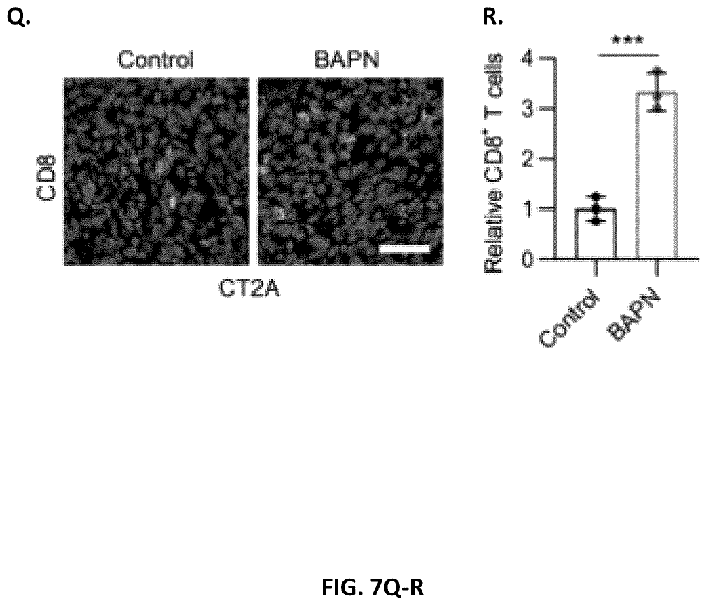

To confirm its role in regulating immune response in vivo, GBM mouse models were developed by intracranial injection of CT2A (PTEN-deficient) or 005 GSC, a GSC line harboring activated AKT, and treated them with LOX neutralizing antibodies or LOX inhibitor β-aminopropionitrile (BAPN), which showed an ability to cross the blood-brain barrier (BBB) (FIG. 7I-K). Immunofluorescence (IF) staining demonstrated that treatment with BAPN or LOX neutralizing antibodies in tumor-bearing C57BL/6 mice increased intratumoral CD4⁺ T cells (FIG. 1H, I and FIG. 7C, D), CD8⁺ T cells (FIGS. 7A, B and Q, R) and activated CD8⁺ (CD8⁺CD69⁺) T cells (FIG. 1F, G and FIG. 7L, M). Given the role of programmed death-ligand 1 (PD-L1) in regulating immunosuppression in GBM, the effect of LOX on PD-L1 expression of PD-L1 in PTEN-deficient GBM cells, such as U87, CT2A, 005 GSC, and PTEN CRISPR KO SF763 cells was further investigated. The results showed that LOX inhibition genetically (e.g., shRNA-mediated LOX depletion) and pharmacologically (treatment with LOX inhibitor BAPN) upregulated the expression of PD-L1 in PTEN-deficient GBM cells (FIG. 7E-H, N-P). Together, these findings led to the hypothesis that LOX inhibition could improve the efficacy of anti-PD1 therapy in PTEN-deficient GBM mouse models. Results showed that BAPN treatment extended the survival of mice bearing CT2A and 005 GSC tumors, and the anti-tumor effect was further augmented when BAPN was combined with anti-PD1 therapy (FIG. 1J, K).

### Example 2

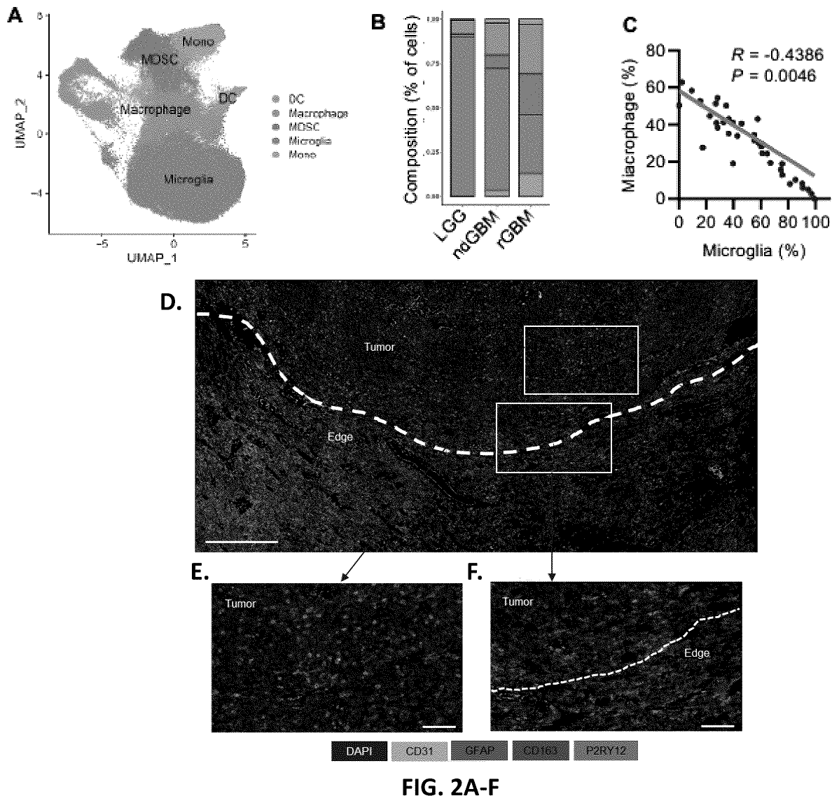

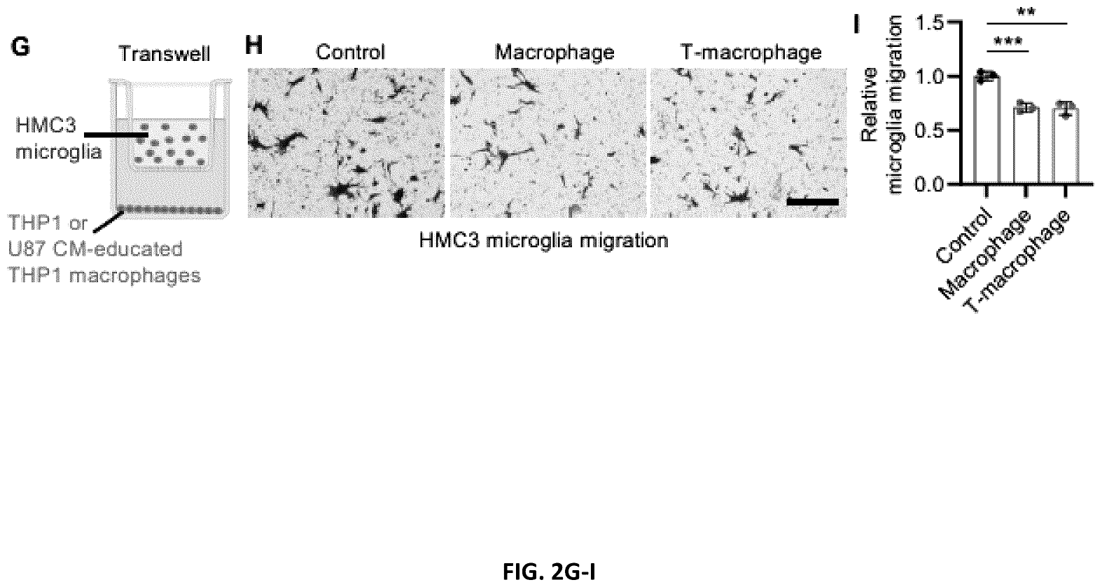

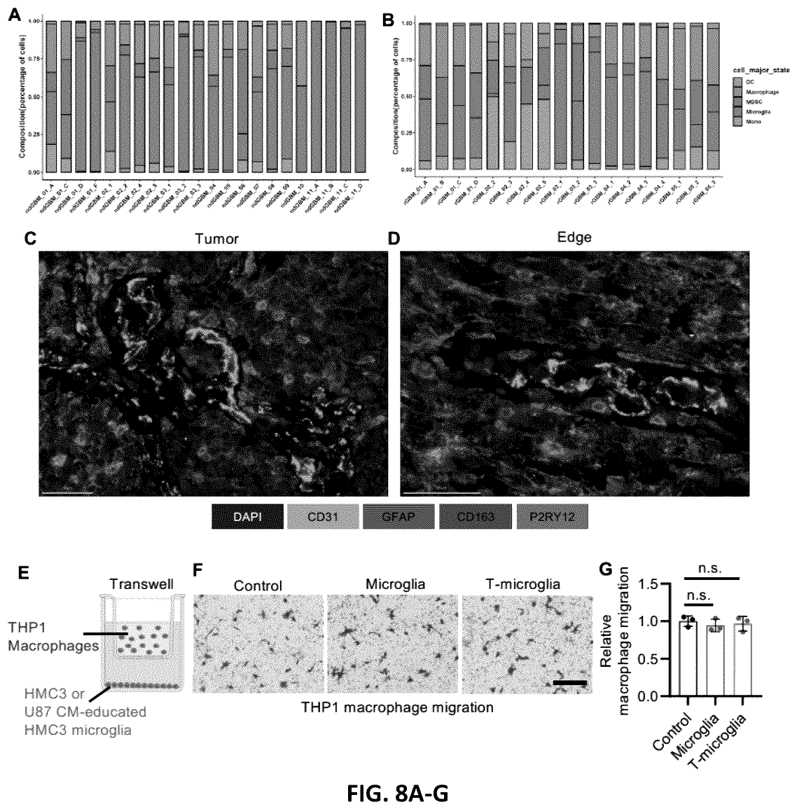

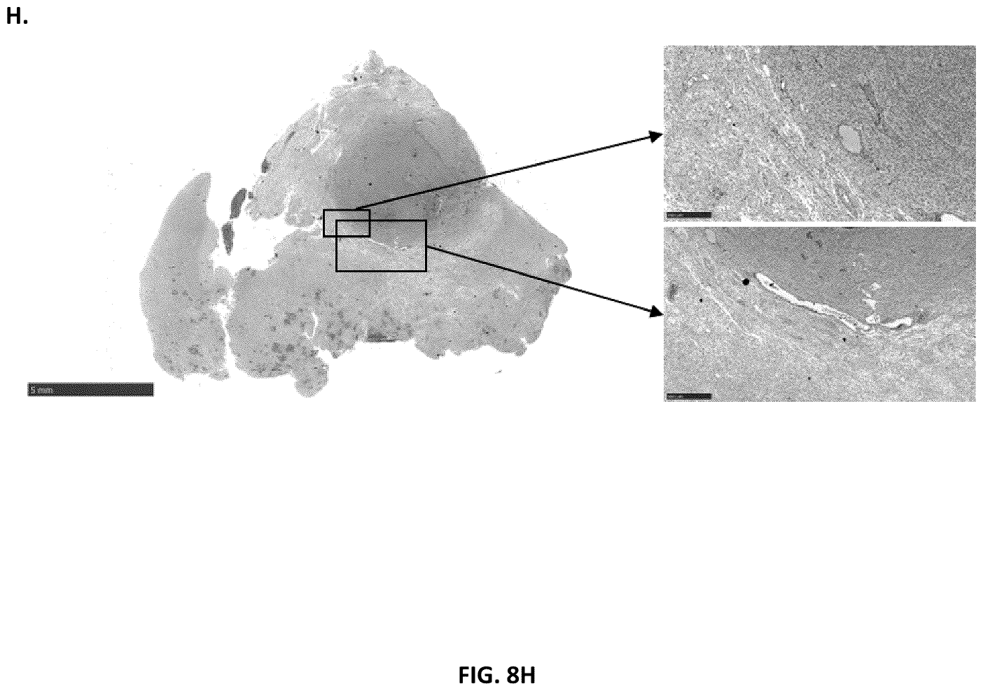

The negative association between macrophages and microglia in the GBM TME. LOX inhibition, both alone and in combination with anti-PD1 therapy, was shown to reduce GBM progression; however, no mice completely cleared their tumors following treatment (FIG. 1J,K). It was further hypothesized that LOX inhibition-induced impairment of macrophage infiltration might induce a compensatory change of other immune cells in the GBM TME. To test this, the scRNA-Seq data from glioma patient tumors was analyzed with a focus on myeloid cells, which include macrophages, microglia, monocytes, dendritic cells (DCs) and myeloid-derived suppressor cells (MDSCs). Among them, macrophages and microglia were the dominant cell populations (FIG. 2A). By analyzing these myeloid cells in low-grade gliomas (LGG), newly diagnosed GBM (ndGBM), and recurrent GBM (rGBM), it was found that macrophage/monocyte density was very low in LGG, increased in ndGBM, and highly enriched in rGBM, whereas microglia showed the opposite expression pattern (FIG. 2B), suggesting a negative correlation between them in glioma patient tumors. Further analysis in GBM patient tumors revealed that the macrophage abundance was negatively correlated with microglia in the TME (FIG. 2C, FIG. 8A, B). Next, multiplex sequential immunofluorescence (SeqIF™) was performed to stain and image whole mount sections of tumors from IDH1-WT GBM patients in continuity with the adjacent brain parenchyma (n=3). The results showed that P2RY12⁺ microglia were mostly distributed in the parenchyma and GBM margin, whereas CD163⁺ macrophages were highly enriched in the tumors (FIG. 2D-F and FIG. 8H). Higher magnified view of tumor sections demonstrated that CD163⁺ macrophages were distributed in the perivascular niches in the tumor and at the brain interface (FIG. 8C, D). More specifically in densely cellular tumor regions, P2RY12⁺ microglia were absent when tumors harbor high abundance of CD163⁺ macrophages (FIG. 2E). CD163⁺ macrophages were relatively low when tumors have high infiltration of P2RY12⁺ microglia (FIG. 2F).

To confirm the functional relationship between macrophages and microglia, transwell migration assays were preformed which showed that macrophages and tumor-educated macrophages (T-macrophage) suppressed the migration of microglia (FIG. 2G-I). Microglia and tumor-educated microglia (T-microglia) had no effect on affecting macrophage migration (FIG. 8E-G). Together, these findings suggested that the infiltration of microglia is suppressed by macrophages in the GBM TME.

### Example 3

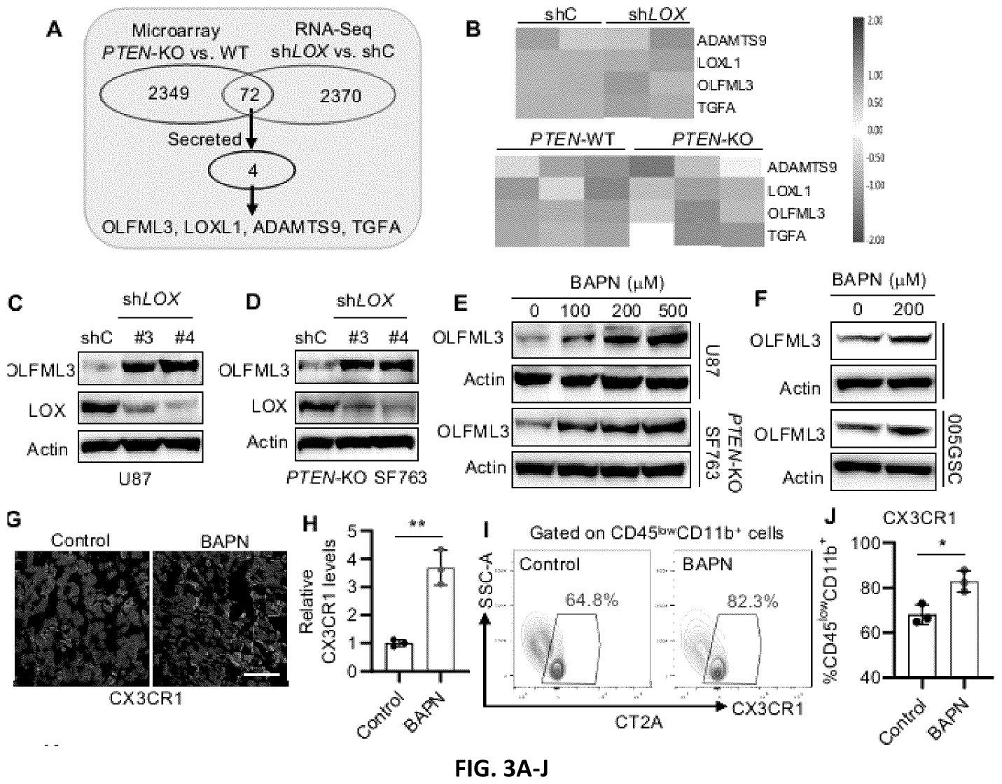

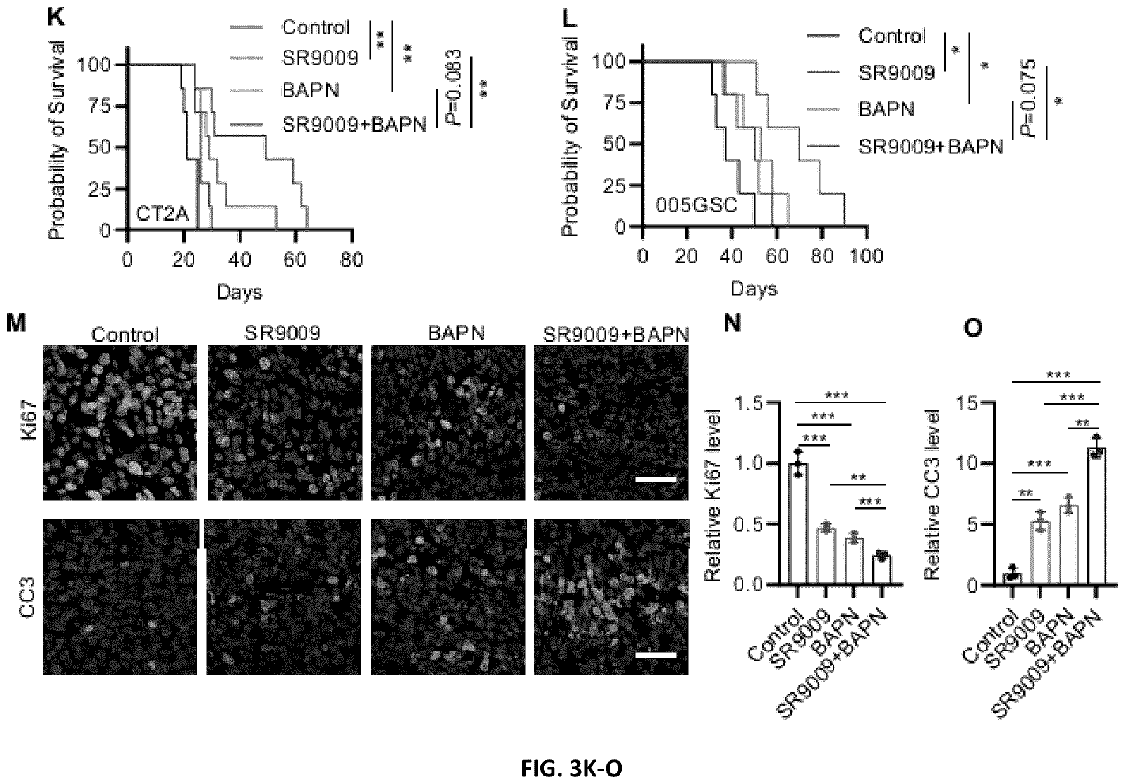

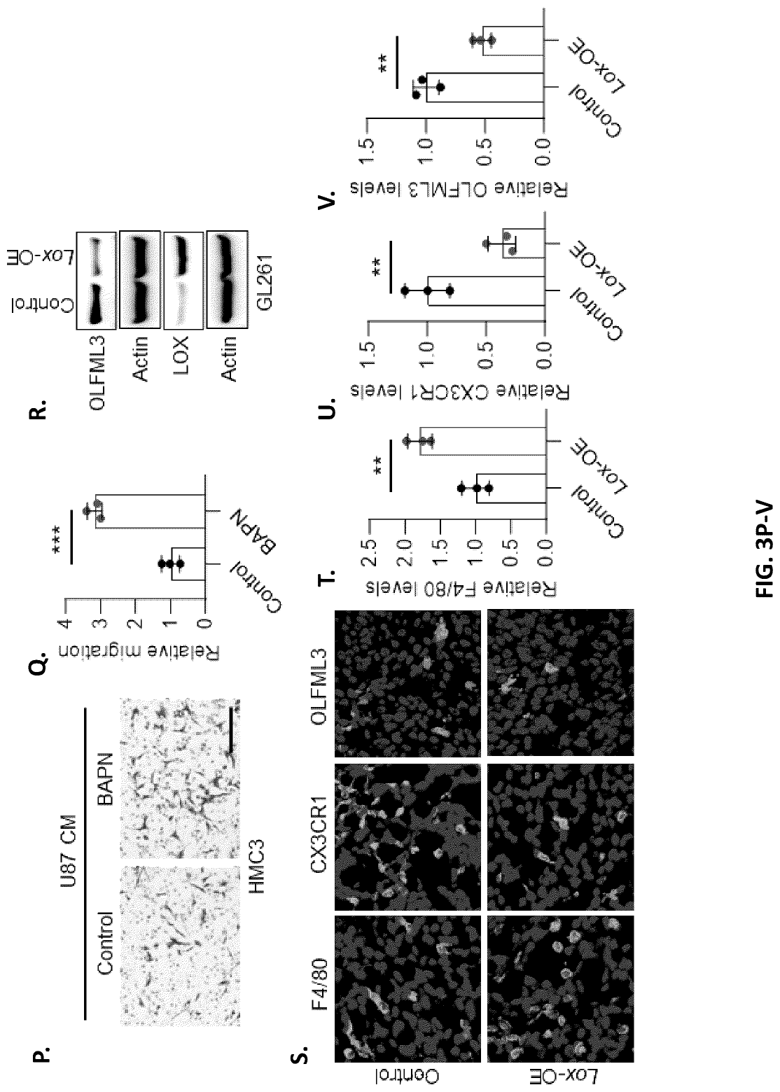

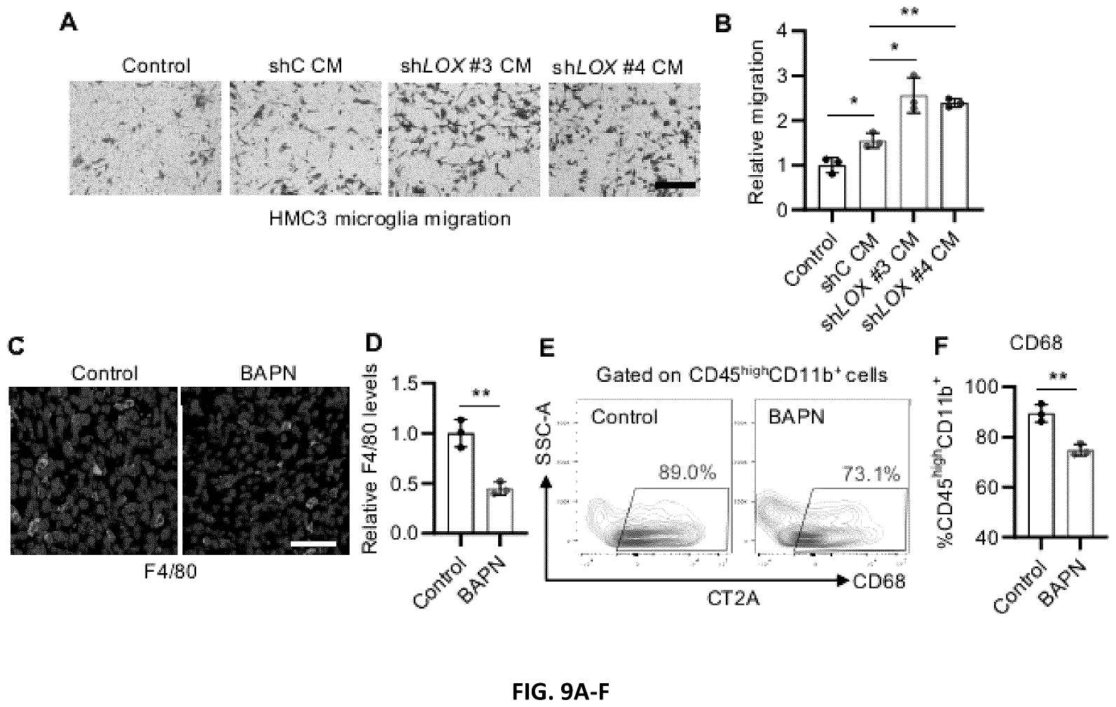

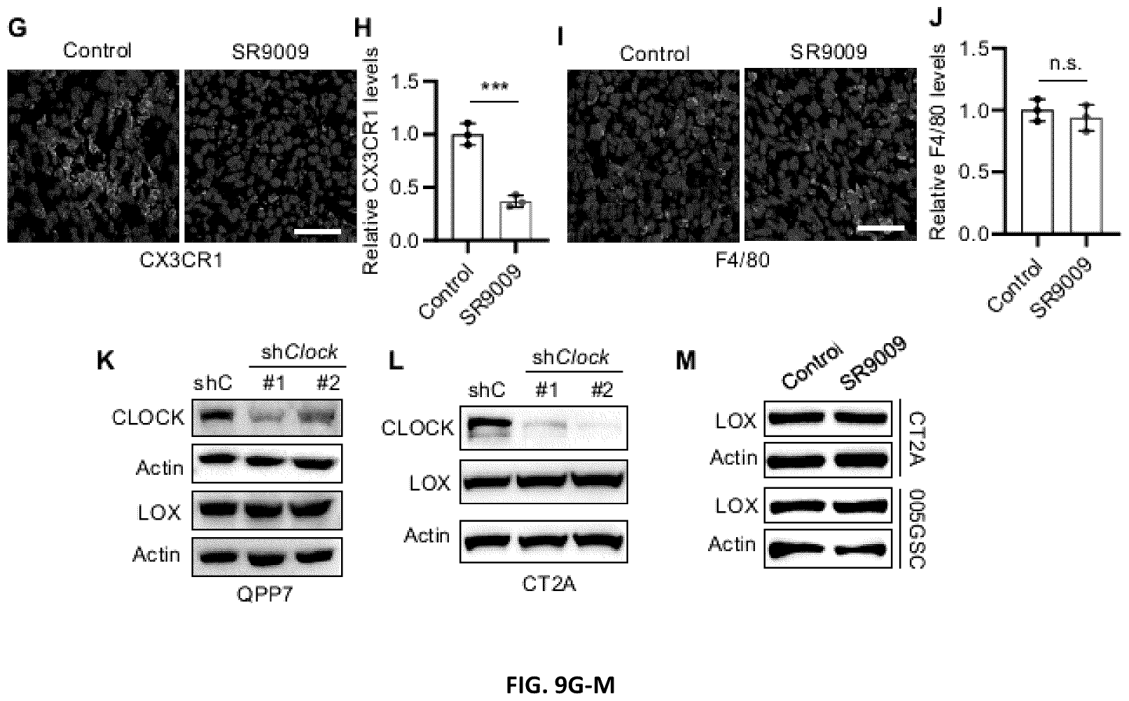

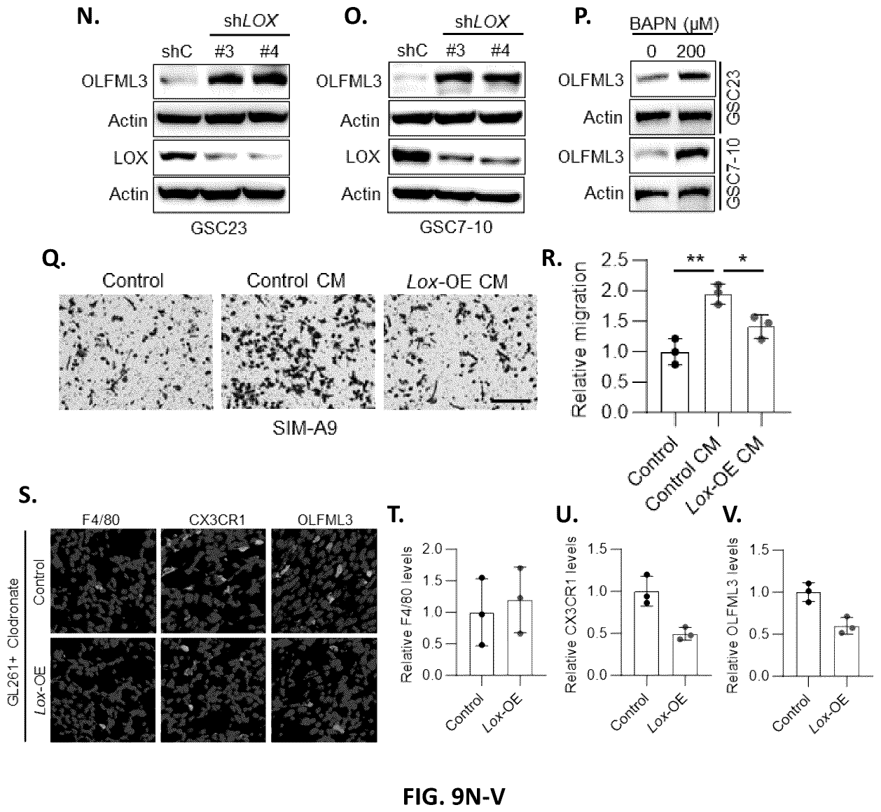

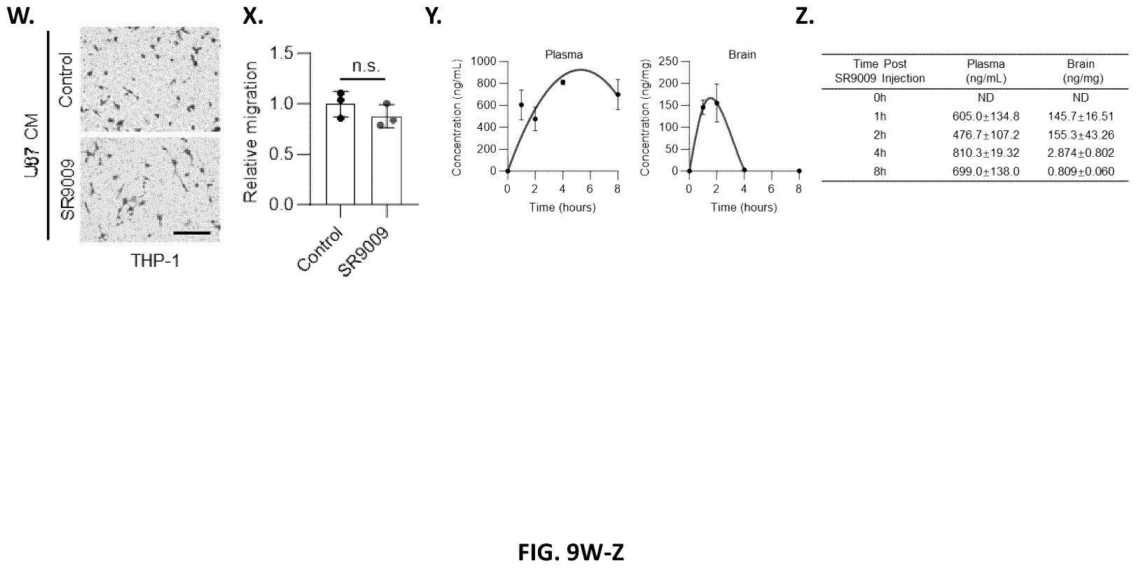

LOX inhibition reduces macrophage infiltration but upregulates OLFML3 expression and microglia infiltration in GBM. Given the critical role of PTEN-LOX signaling axis in triggering macrophage infiltration, it was investigated whether LOX inhibition can induce compensatory changes of chemokines that might affect microglia infiltration. To this end, performed was an RNA-Seq profiling in U87 cells with LOX shRNA (shLOX) versus shRNA control (shC). By analyzing the RNA-Seq data as well as microarray data of SF763 cells with PTEN-KO versus WT, identified four genes (e.g., OLFML3, LOXL1, ADAMTS9, and TGFA) that were upregulated by LOX knockdown and downregulated by PTEN KO in GBM cells (FIG. 3A, B). Among them, OLFML3 was important since findings have shown that OLFML3 is a microglia chemokine in CLOCK-high GBM. Immunoblotting results confirmed that shRNA-mediated LOX knockdown in PTEN-deficient GBM cells (e.g., U87 and PTEN-KO SF763 cells) and GSCs (e.g., GSC23 and GSC7-10) upregulated OLFML3 expression (FIG. 3C, D and FIG. 9N, O). Similarly, pharmacologic inhibition of LOX using the inhibitor BAPN increased the expression of OLFML3 in both human (e.g., U87, PTEN-KO SF763, GSC23, and GSC7-10) and mouse (e.g., CT2A and 005 GSCs) GBM cells and GSCs (FIG. 3E, F and FIG. 9P).

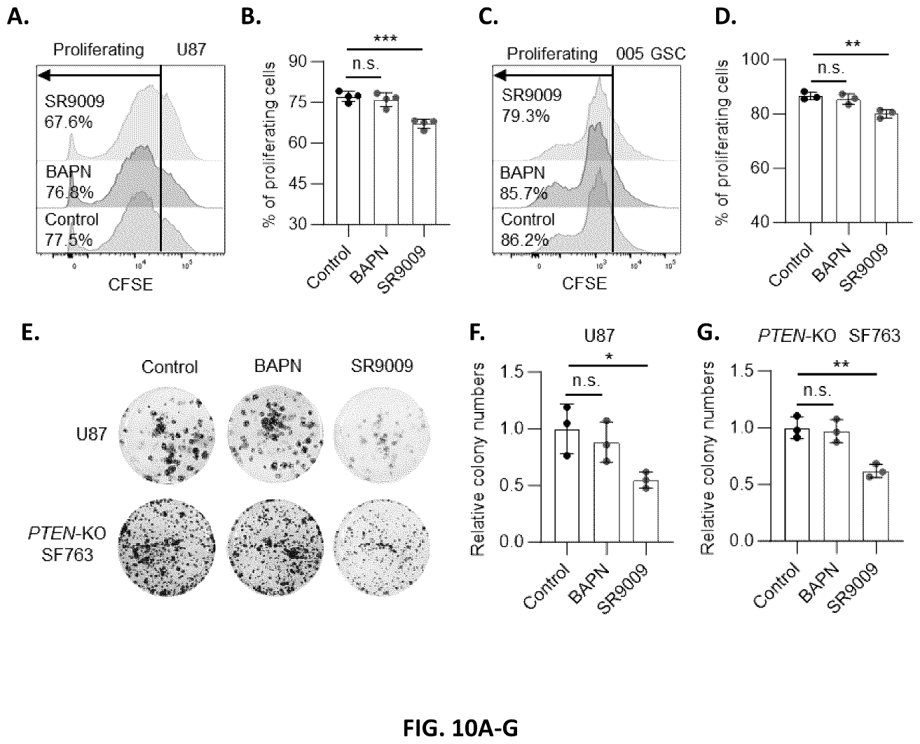

To confirm whether LOX inhibition-induced upregulation of OLFML3 could affect microglia infiltration in the GBM TME, the first performed was transwell migration assays with results showing that the conditioned media (CM) from LOX-inhibited/depleted U87 cells increased the migration ability of HMC3 microglia (FIG. 3P, Q and FIG. 9A, B). LOX in PTEN-WT GL261 cells were overexpressed (FIG. 3R) and then checked OLFML3 expression in control and LOX-overexpressed (Lox-OE) cells and used CM from them to perform transwell migration assay. The results showed that LOX overexpression downregulated OLFML3 expression in GBM cells (FIG. 3R) and reduced the migration of SIM-A9 microglia (FIG. 9Q, R). In addition to these in vitro studies, microglia and macrophage populations were analyzed in control, LOX-inhibited, and LOX-overexpressed tumors. Additionally, microglia populations were analyzed in control and LOX-inhibited CT2A tumors. The results from immunofluorescence staining showed that BAPN-treated CT2A tumors had higher CX3CR1⁺ microglia (FIG. 3G-H) and lower F4/80⁺ macrophages (FIG. 9C, D) compared to control tumors. Flow cytometry analysis showed that BAPN treatment increased CD45^{low}CD11b⁺CX3CR1⁺ microglia and decreased CD45^{high}CD11b⁺CD68⁺ macrophages in CT2A tumors (FIG. 3I, J and FIG. 9E, F). In contrast, LOX overexpression induced higher infiltration of macrophages and lower OLFML3 expression and microglia infiltration in the GBM TME (FIG. 3S-V). And LOX overexpression in GL261 cells still downregulated OLFML3 expression and microglia infiltration in macrophage-depleted tumors (FIG. 9S-V). Consistent with these findings, the inhibition of CLOCK using the Rev-ErbA agonist SR9009 reduced CX3CR1⁺ microglia (FIGS. 9G, H and Y, Z). However, SR9009 treatment did not affect F4/80⁺ macrophages (FIG. 9I, J). Accordingly, knockdown of CLOCK or treatment with SR9009 did not affect LOX expression in QPP7 GSCs (PTEN-deficient), CT2A cells and 005 GSCs (FIG. 9K-M). And the CM from SR9009-treated U87 cells did not change the migration ability of THP-1 macrophages (FIG. 9W, X). Further confirmed was that SR9009 treatment impaired GBM cell proliferation, but LOX inhibition using BAPN had no such effect (FIG. 10A-G). These findings lead to the development of a novel therapeutic strategy by targeting the compensatory mechanism between macrophages and microglia via simultaneously inhibiting LOX and CLOCK-OLFML3 axis. A proof-of-principal was conducted in combination of BAPN and SR9009 in GBM-bearing mice; it was found that there was significant survival extension relative to monotherapy in both CT2A and 005 GSC models (FIG. 3K, L). On the histological level, proliferation marker Ki67 was dramatically decreased, whereas apoptosis marker cleaved caspase 3 (CC3) was significantly increased in BAPN and SR9009 combination treatment group compared to single treatment and control groups (FIG. 3M-O).

### Example 4

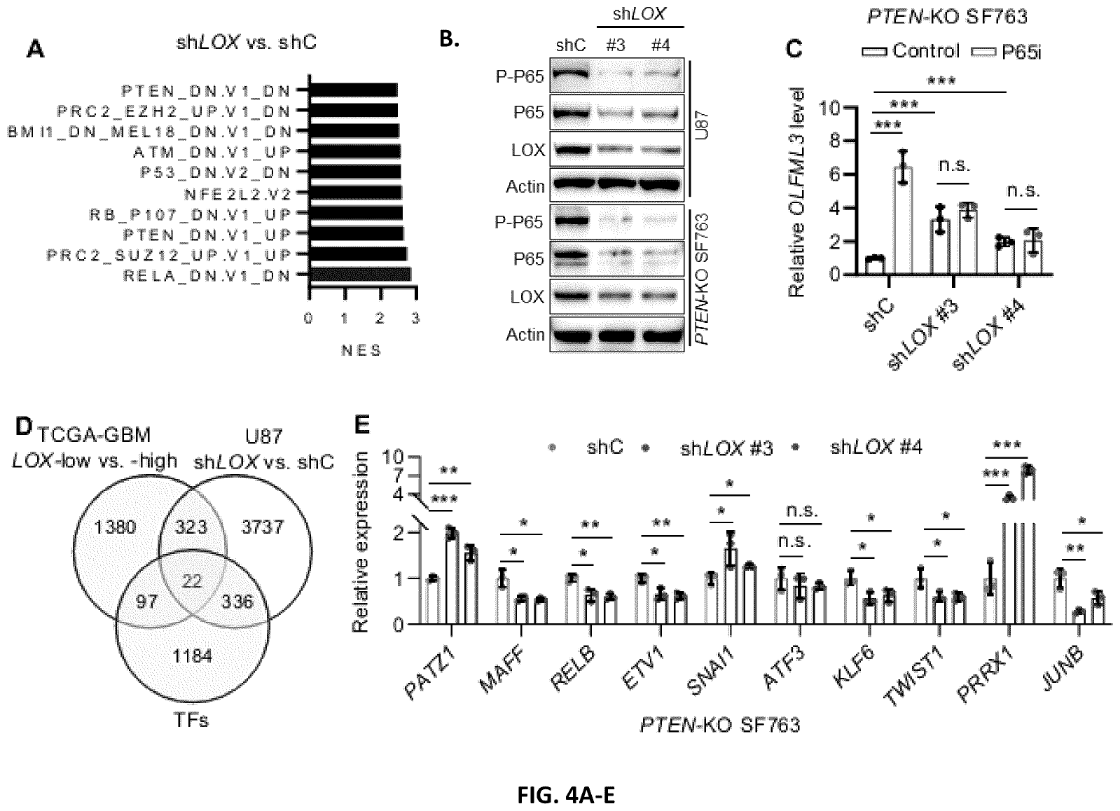

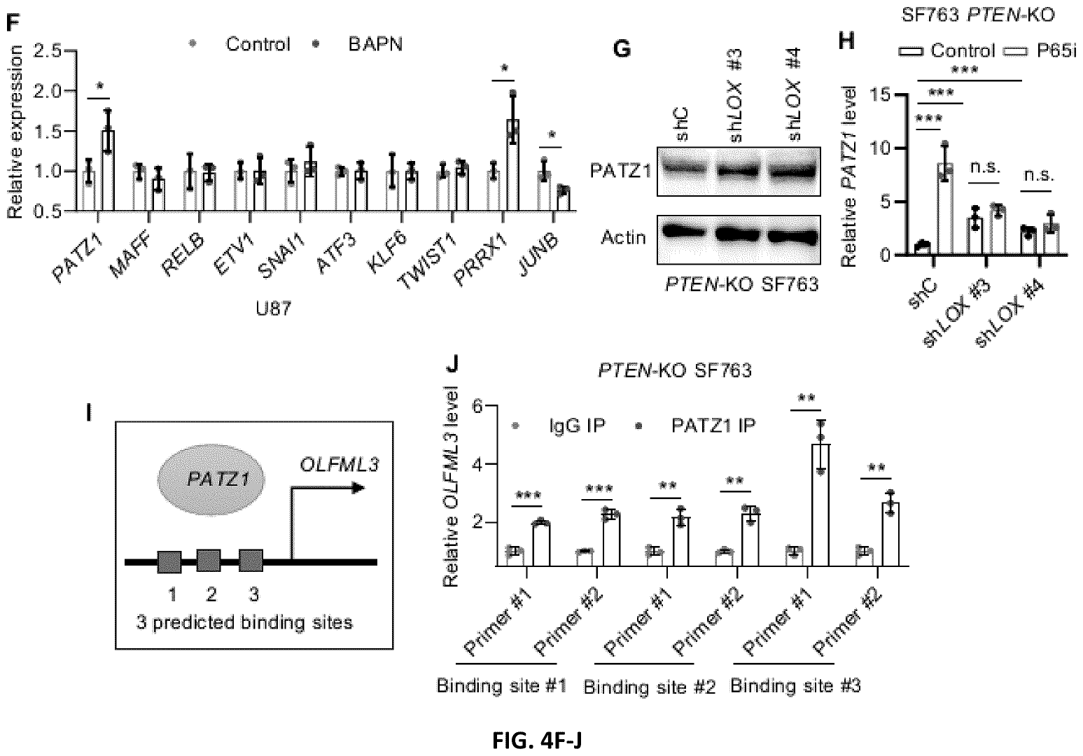

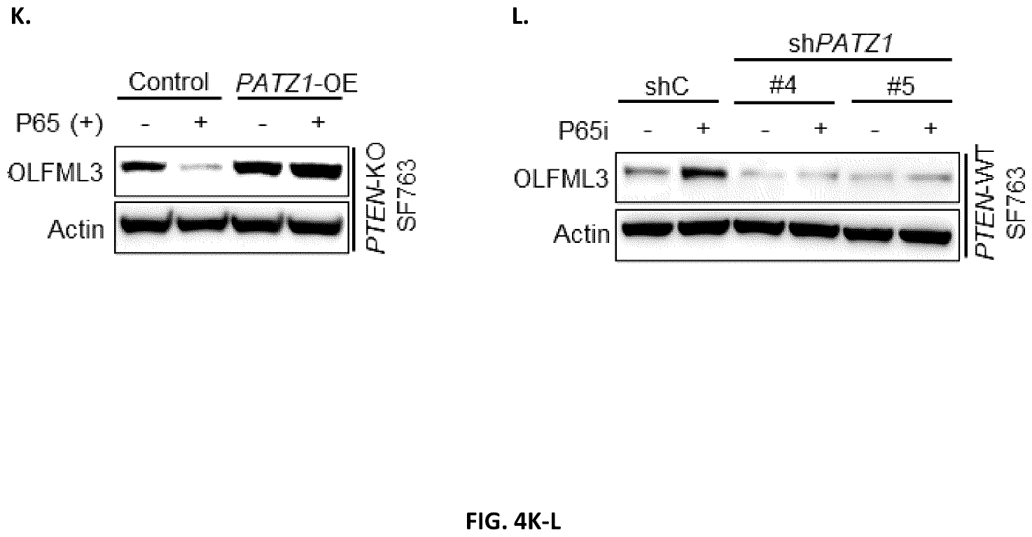

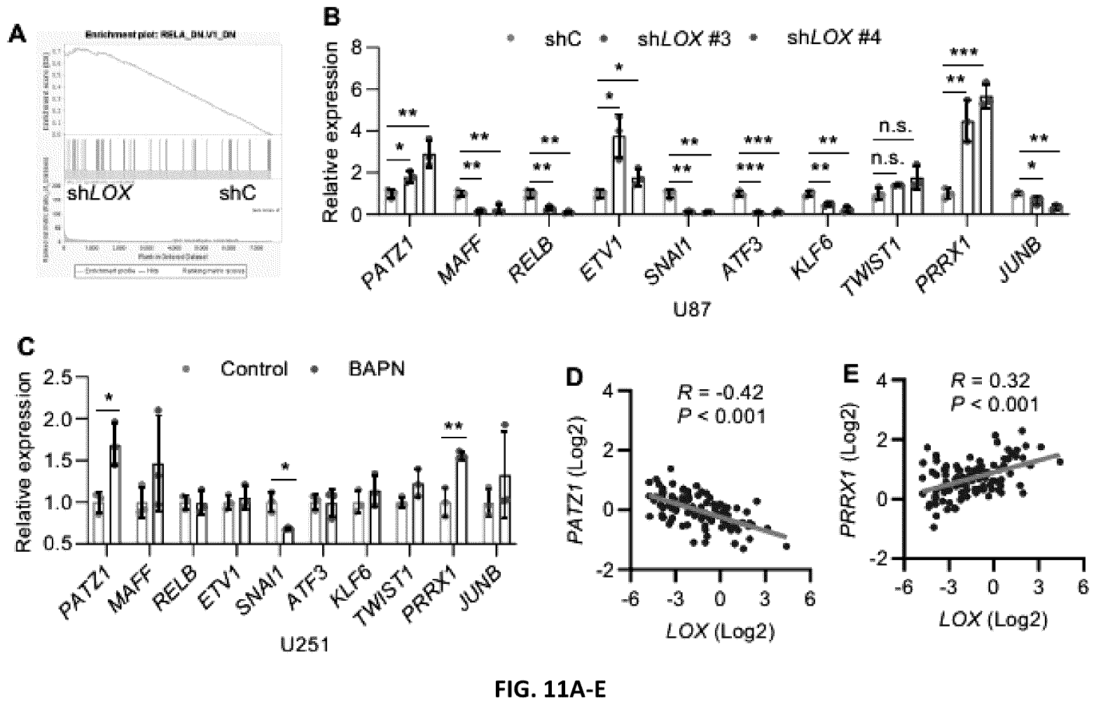

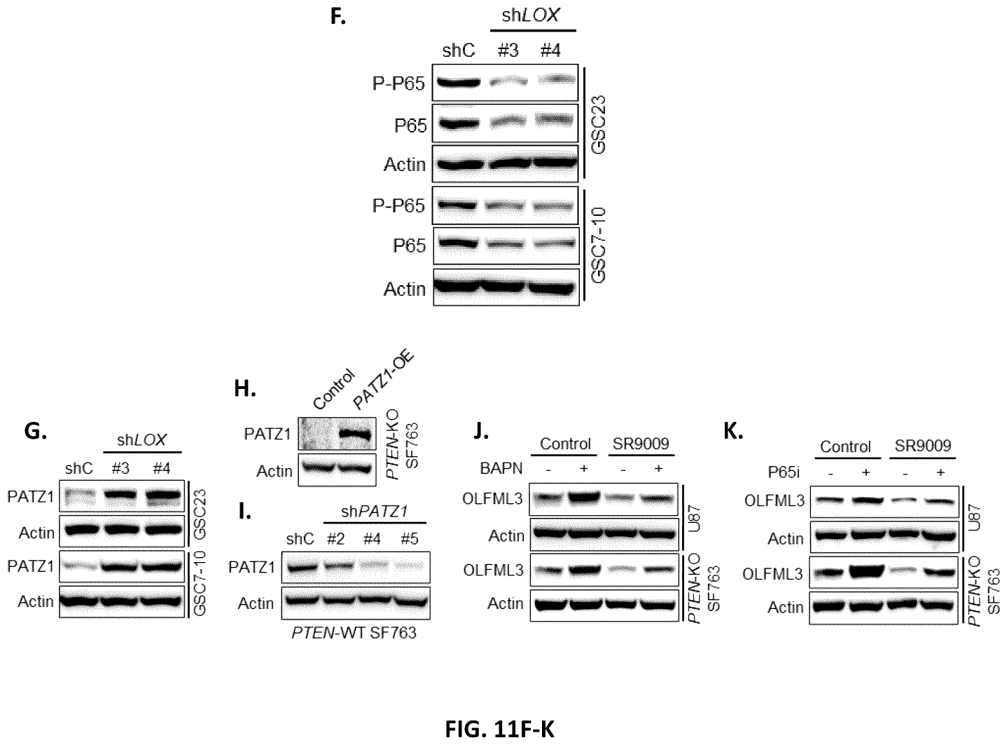

LOX affects OLFML3 expression via regulating the NF-kB-PATZ1 signaling axis. To explore the potential mechanism for how LOX regulates OLFML3, GSEA was used to catalog oncogenic signaling pathways modulated by LOX in U87 cells (shLOX versus shC). The RELA_DN.v1_DN was identified as the top signature affected by LOX (FIG. 4A and FIG. 11A), suggesting the importance of LOX in regulating NF-kB pathway. The results from Western blotting demonstrated that shRNA-mediated depletion of LOX in U87, PTEN-KO SF763, GSC23, and GSC7-10 cells significantly inhibited the NF-kB subunit P65 and Phospho-P65 (FIG. 4B and FIG. 11F). To investigate the potential functional relevance of P65 in regulating OLFML3 expression in GBM cells, shC and shLOX PTEN-KO SF763 cells were treated with the P65 inhibitor SC75741. The results showed that inhibition of P65 upregulated the expression of OLFML3 in shC cells (FIG. 4C).

To further identify LOX-regulated factors that can transcriptionally regulate OLFML3 in PTEN-null GBM cells, differential expressed genes encoding human transcriptional factors (TFs) in U87 cells were overlapped with shLOX versus shC and in TCGA GBM tumors with LOX-low versus LOX-high. As a result, 22 potential TFs were identified (FIG. 4D), which were inserted into the JASPAR database with results showing that 10 of them can potentially bind to the OLFML3 promoter. The results from RT-qPCR assays in PTEN-null GBM cells, such as U87, PTEN-KO SF763 and U251 cells, revealed that PATZ1 and PRRX1 were upregulated upon shRNA-mediated LOX depletion and the treatment with LOX inhibitor BAPN (FIG. 4E, F and FIG. 11B, C). Bioinformatics analyses in TCGA GBM patient tumors demonstrated that PATZ1 correlated negatively with LOX, whereas PRRX1 showed a positive correlation with LOX (FIG. 11D, E). The results from Western blotting confirmed that depletion of LOX upregulated PATZ1 protein level in PTEN-KO SF763, GSC23, and GSC7-10 cells (FIG. 4G and FIG. 11G). The next aim was to confirm whether PATZ1 is regulated by P65 and whether PATZ1 can bind to the promotor of OLFML3 in PTEN-null GBM cells. RT-qPCR demonstrated that P65 inhibition upregulated the expression of PATZ1 in shC, but not shLOX PTEN-KO SF763 cells (FIG. 4H), suggesting that PATZ1 is a downstream TF of the NF-kB pathway. Based on the predicted binding sites (FIG. 4I), 6 pairs of primers were designed and performed ChIP-PCR assays with results showing that PATZ1 bound to the OLFML3 promoter in PTEN-KO SF763 cells (FIG. 4J).

To further validate the function of NF-kB-PATZ1 signaling axis in regulating OLFML3 in GBM cells, PATZ1 in PTEN-KO SF763 cells were overexpressed (FIG. 11H) and found that PATZ1 overexpression enhanced OLFML3 expression and abolished P65 activation-induced downregulation of OLFML3 (FIG. 4K). Conversely, shRNA-mediated PATZ1 depletion in PTEN-WT SF763 cells negated P65 inhibition-induced upregulation of OLFML3 (FIG. 4L and FIG. 11I). OLFML3 can be transcriptionally regulated by CLOCK in GBM therefore, it was investigated whether the regulatory effect of LOX-NF-kB-PATZ1 signaling axis on OLFML3 transcription was independent on CLOCK. Immunoblotting results showed that LOX or P65 inhibition-induced OLFML3 upregulation was rescued by the treatment with SR9009 (FIG. 11J, K). Together, these findings suggest that inhibition of LOX upregulates OLFML3 via regulating the NF-kB-PATZ1 signaling axis in PTEN-null GBM cells.

### Example 5

Dual inhibition of LOX and CLOCK-OLFML3 axis activates anti-tumor immune response and synergizes with anti-PD1 therapy.

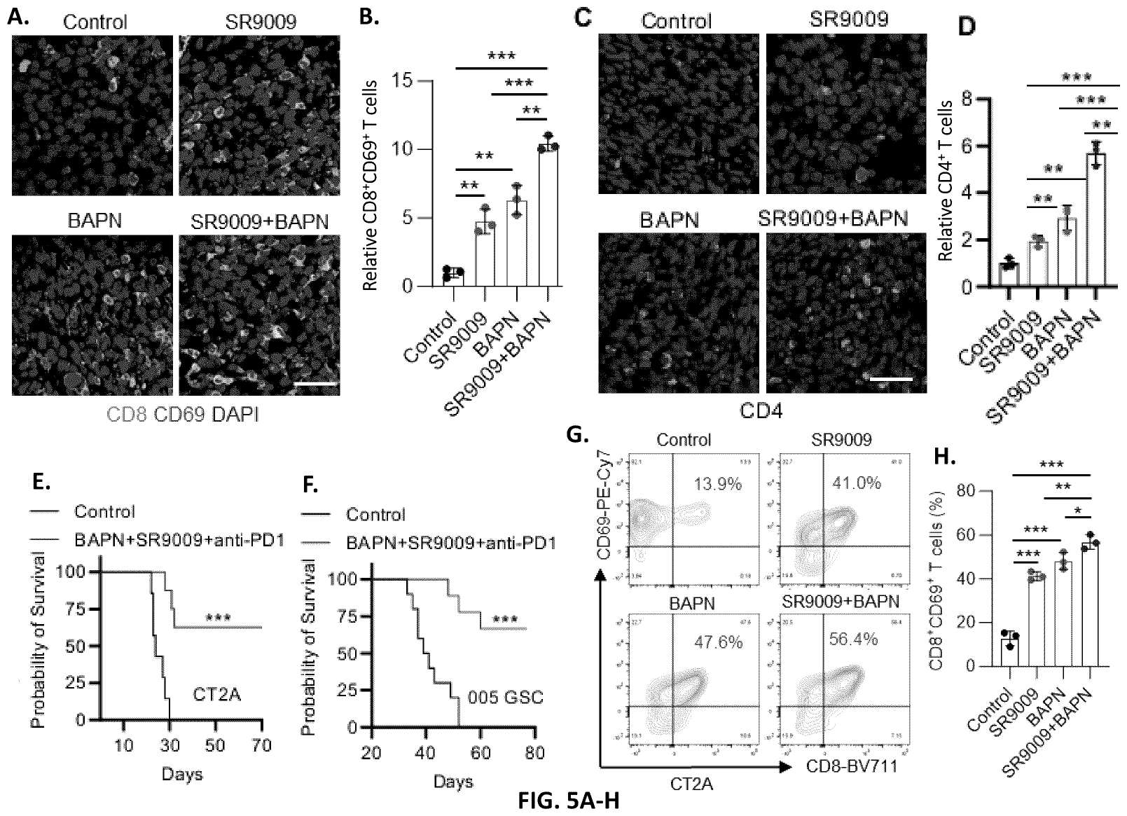

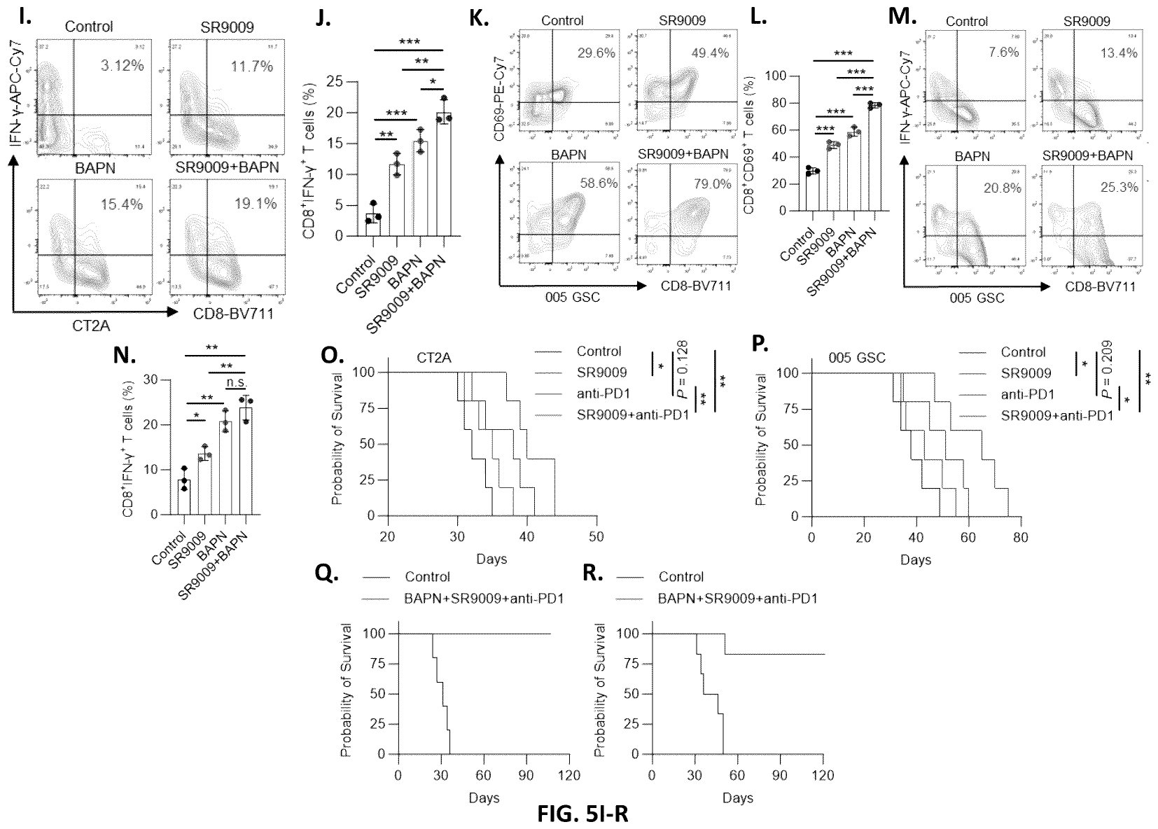

Similar to the survival benefits induced by LOX inhibition (FIG. 1J, K), it was found that CLOCK inhibition using SR9009 combined with anti-PD1 therapy resulted in survival extension, but did not cure any tumor-bearing mice, in CT2A and 005 GSC models (FIG. 5O, P). Given the compensatory upregulation of microglia upon LOX inhibition, it was hypothesized that dual targeting macrophages and microglia using BAPN and SR9009 would produce potent anti-tumor immunity in PTEN-deficient GBM. Immunofluorescence staining demonstrated that intratumoral CD8⁺ T cells (FIG. 5A, B), CD4⁺ T cells (FIG. 5C, D) and activated (CD69⁺) T cells (FIG. 5A, B) were increased upon the treatment with BAPN or SR9009, and these enhancements were further heightened when these two treatments were combined. Increases of activated CD8⁺ T cells induced by the treatment with BAPN, SR9009 and their combination were confirmed by flow cytometry for CD45⁺CD3⁺CD8⁺CD69⁺ and CD45⁺CD3⁺CD8⁺IFN-γ⁺ activated T cells in both CT2A and 005 GSC tumors (FIG. 5G-N). In preclinical trials, the triple therapy with BAPN, SR9009 and anti-PD1 resulted in a significant survival extension in both CT2A (FIG. 5E) and 005 GSC models (FIG. 5F). Notably, 63% and 67% of CT2A and 005 GSC tumor-bearing mice cleared their tumors after the therapy (FIG. 5E, F). The triple therapy (BAPN+SR9009+anti-PD1) activated T cell memory, as almost all the mice that had previously cleared GBM tumors efficiently suppressed tumor growth when re-challenged with CT2A cells or 005 GSCs and remained tumor-free (FIG. 5Q, R). Together, these findings suggest that the triple therapy targeting macrophage and microglia infiltration, combined with anti-PD1 therapy, is a promising therapeutic strategy for PTEN-deficient GBM.

### Example 6

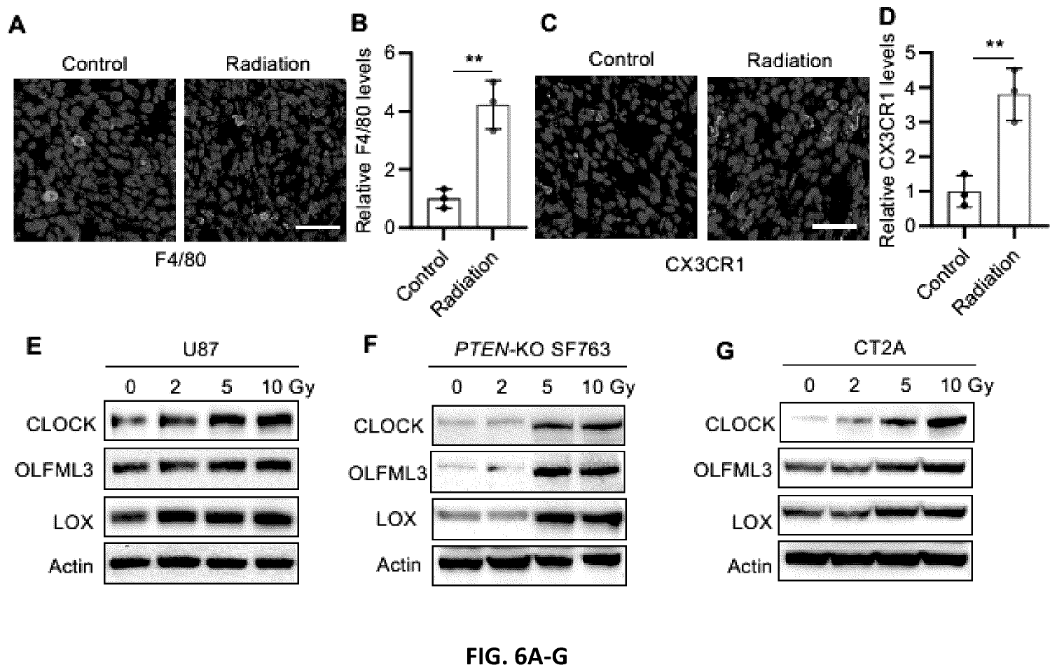

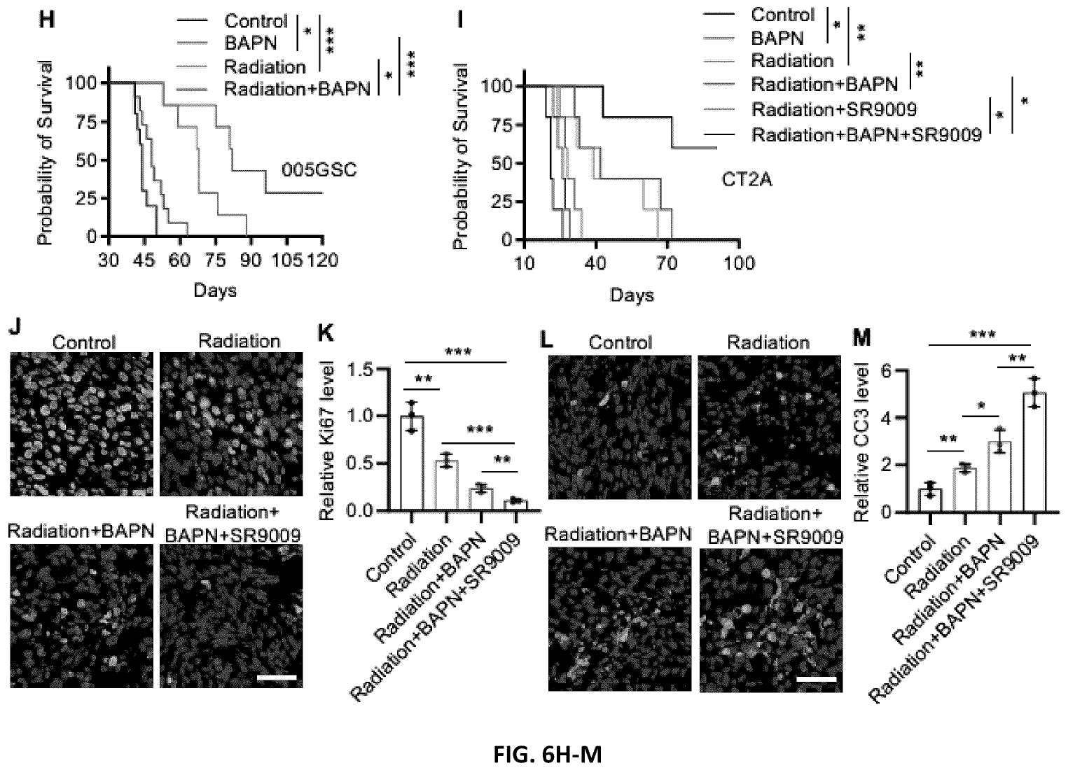

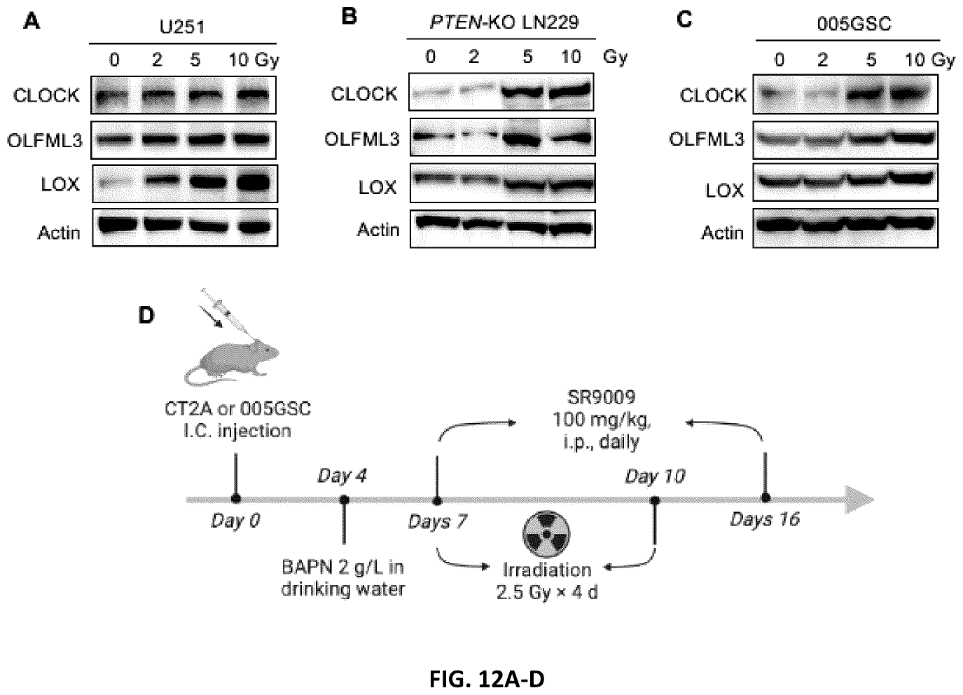

Dual inhibition of LOX and CLOCK-OLFML3 axis improves the anti-tumor efficiency of radiotherapy. Radiotherapy resistance always occurs in GBM patients, in part, due to TME heterogeneity. Immunofluorescence staining revealed that radiation treatment led to an increase of F4/80⁺ macrophages and CX3CR1⁺ microglia in CT2A tumors (FIG. 6A-D), which indicated that there was need for further exploration of their roles in regulating radiotherapy efficiency in GBM. Considering the roles of LOX and the CLOCK-OLFML3 axis in promoting the infiltration of macrophages and microglia, respectively, the expression levels of LOX, CLOCK and OLFML3 in control and irradiated PTEN-deficient GBM cells were further evaluated. The results showed that radiation upregulated the expression of LOX, CLOCK and OLFML3 in a dose-dependent manner in both human (e.g., U87, PTEN-KO SF763, U251, and PTEN-KO LN229) and mouse (CT2A and 005 GSCs) GBM cells (FIG. 6E-G and FIG. 12A-C). Based on these results, a combination treatment strategy using radiation, BAPN, and/or SR9009 in PTEN-deficient GBM mouse models was designed (FIG. 12D). It was found that radiation combined with BAPN treatment significantly improved the survival of 005 GSC tumor-bearing mice and 25% of mice were long-term survivors (FIG. 6H). Similarly, radiation combined with BAPN or SR9009 treatment in CT2A model resulted in significant survival extension (FIG. 6I). Notably, the survival was further prolonged in CT2A-bearing mice when they received the triple therapy with radiation, BAPN and SR9009, and 60% of animal subjects were long-term survivors (FIG. 6I). On the histological level, the triple therapy significantly reduced Ki67 and upregulated CC3 compared to radiation alone or combined with BAPN (FIG. 6J-M).

1. A dual targeting anti-tumor composition comprising
a. a LOX inhibitor; and
b. a CLOCK-OLFML3 inhibitor (CLOCK-OLFML3 axis blockade).

2. The dual targeting anti-tumor composition of claim 1, wherein the LOX inhibitor suppresses macrophage infiltration and upregulates microglial infiltration.

3. The dual targeting anti-tumor composition of claim 1, wherein the LOX inhibitor upregulates expression of Programmed Death-Ligand 1 (PD-L1) and OLFML3.

4. The dual targeting anti-tumor composition of claim 1, wherein the LOX inhibitor modulates OLFML3 expression via the NF-kB-PATZ1 signaling axis.

5. The dual targeting anti-tumor composition of claim 1, wherein the LOX inhibitor is a β-aminopropionitrile (BAPN), BAPN derivative, or an aminomethylenethiophene (AMT).

6. The dual targeting anti-tumor composition of claim 1, wherein the LOX inhibitor is an inhibitor of LOX expression.

7. The dual targeting anti-tumor composition of claim 1, wherein the CLOCK-OLFML3 inhibitor suppresses microglial infiltration.

8. The dual targeting anti-tumor composition of claim 1, wherein the CLOCK-OLFML3 inhibitor suppresses OLFML3 expression.

9. The dual targeting anti-tumor composition of claim 1, wherein the CLOCK-OLFML3 inhibitor is a SR9009.

10. The dual targeting anti-tumor composition of claim 1, wherein the CLOCK-OLFML3 inhibitor is an inhibitor of CLOCK-OLFML3 expression.

11. The dual targeting anti-tumor composition of claim 1, wherein the LOX inhibitor and the CLOCK-OLFML3 inhibitor are administered simultaneously or independently.

12. A method for treating cancer in a subject comprising administering a dual targeting anti-tumor composition of one of claim 1 to the subject.

13. The method of claim 12, further comprising co-administering an effective amount of an additional cancer therapeutic to the subject.

14. The method of claim 13, wherein the dual targeting anti-tumor composition and the additional cancer therapeutic are administered simultaneously.

15. The method of claim 13, wherein the dual targeting anti-tumor composition and the additional cancer therapeutic are administered independently.

16. The method of claim 12, wherein the cancer is glioblastoma (GBM).

17. The method of claim 16, wherein the GBM is PTEN-deficient.

18. The method of claims 13, wherein the additional cancer therapeutic is an Anti-Programmed Death 1 (PD1) therapy.

19. The method of claims 13, wherein the additional cancer therapeutic is a radiotherapy.

20. The method of claims 12, wherein the subject is a human.


SEQUENCE LISTING (36 sequences)

SEQ ID NO: 1 - DNA, 21 bp, Synthetic construct
  source 1..21: mol_type = other DNA
tccttttgtcatggtcgggac

SEQ ID NO: 2 - DNA, 21 bp, Synthetic construct
  source 1..21: mol_type = other DNA
taaagcagctagtcggcgttc

SEQ ID NO: 3 - DNA, 22 bp, Synthetic construct
  source 1..22: mol_type = other DNA
gatgcacactatcagctccaag

SEQ ID NO: 4 - DNA, 21 bp, Synthetic construct
  source 1..21: mol_type = other DNA
cgataaccgacctcatcagca

SEQ ID NO: 5 - DNA, 19 bp, Synthetic construct
  source 1..19: mol_type = other DNA
tgcccaggtcccatttctc

SEQ ID NO: 6 - DNA, 18 bp, Synthetic construct
  source 1..18: mol_type = other DNA
ggcccacgaagggaatgt

SEQ ID NO: 7 - DNA, 19 bp, Synthetic construct
  source 1..19: mol_type = other DNA
cagcctcgtggggaaagac

SEQ ID NO: 8 - DNA, 22 bp, Synthetic construct
  source 1..22: mol_type = other DNA
gcccaggttgttaaaactgtgc

SEQ ID NO: 9 - DNA, 21 bp, Synthetic construct
  source 1..21: mol_type = other DNA
ggccccaggcagttttatgat

SEQ ID NO: 10 - DNA, 20 bp, Synthetic construct
  source 1..20: mol_type = other DNA
gatcctcgccgttggtatgt

SEQ ID NO: 11 - DNA, 21 bp, Synthetic construct
  source 1..21: mol_type = other DNA
tcggaagcctaactacagcga

SEQ ID NO: 12 - DNA, 20 bp, Synthetic construct
  source 1..20: mol_type = other DNA
agatgagcattggcagcgag

SEQ ID NO: 13 - DNA, 20 bp, Synthetic construct
  source 1..20: mol_type = other DNA
cctctgcgctggaatcagtc

SEQ ID NO: 14 - DNA, 22 bp, Synthetic construct
  source 1..22: mol_type = other DNA
ttctttctcgtcgcctcttttt

SEQ ID NO: 15 - DNA, 20 bp, Synthetic construct
  source 1..20: mol_type = other DNA
ggcaacagacctgcctagag

SEQ ID NO: 16 - DNA, 21 bp, Synthetic construct
  source 1..21: mol_type = other DNA
ctcccgagccagaatgatttt

SEQ ID NO: 17 - DNA, 20 bp, Synthetic construct
  source 1..20: mol_type = other DNA
gtccgcagtcttacgaggag

SEQ ID NO: 18 - DNA, 22 bp, Synthetic construct
  source 1..22: mol_type = other DNA
gcttgagggtctgaatcttgct

SEQ ID NO: 19 - DNA, 19 bp, Synthetic construct
  source 1..19: mol_type = other DNA
caggcggatgagaacgtgg

SEQ ID NO: 20 - DNA, 23 bp, Synthetic construct
  source 1..23: mol_type = other DNA
aaaagcatcaggatagtgtgtcc

SEQ ID NO: 21 - DNA, 21 bp, Synthetic construct
  source 1..21: mol_type = other DNA
acgactcatacacagctacgg

SEQ ID NO: 22 - DNA, 23 bp, Synthetic construct
  source 1..23: mol_type = other DNA
gctcggtttcaggagtttgtagt

SEQ ID NO: 23 - DNA, 21 bp, Synthetic construct
  source 1..21: mol_type = other DNA
ggagcgagatccctccaaaat

SEQ ID NO: 24 - DNA, 23 bp, Synthetic construct
  source 1..23: mol_type = other DNA
ggctgttgtcatacttctcatgg

SEQ ID NO: 25 - DNA, 19 bp, Synthetic construct
  source 1..19: mol_type = other DNA
gaggtggttaaggcttctg

SEQ ID NO: 26 - DNA, 20 bp, Synthetic construct
  source 1..20: mol_type = other DNA
ctctcactactctggaatgc

SEQ ID NO: 27 - DNA, 19 bp, Synthetic construct
  source 1..19: mol_type = other DNA
gaggtggttaaggcttctg

SEQ ID NO: 28 - DNA, 20 bp, Synthetic construct
  source 1..20: mol_type = other DNA
ctcactactctggaatgctt

SEQ ID NO: 29 - DNA, 18 bp, Synthetic construct
  source 1..18: mol_type = other DNA
cctgcttgactgcctaga

SEQ ID NO: 30 - DNA, 18 bp, Synthetic construct
  source 1..18: mol_type = other DNA
agccagaagccttaacca

SEQ ID NO: 31 - DNA, 18 bp, Synthetic construct
  source 1..18: mol_type = other DNA
cctgcttgactgcctaga

SEQ ID NO: 32 - DNA, 18 bp, Synthetic construct
  source 1..18: mol_type = other DNA
gcagccagaagccttaac

SEQ ID NO: 33 - DNA, 20 bp, Synthetic construct
  source 1..20: mol_type = other DNA
ggtgaagtctaagccttagt

SEQ ID NO: 34 - DNA, 19 bp, Synthetic construct
  source 1..19: mol_type = other DNA
ggtcaagcccaacttattc

SEQ ID NO: 35 - DNA, 20 bp, Synthetic construct
  source 1..20: mol_type = other DNA
ggtgaagtctaagccttagt

SEQ ID NO: 36 - DNA, 18 bp, Synthetic construct
  source 1..18: mol_type = other DNA
aagggaggaagcaggaaa